(19) United States
(12) Patent Application Publication
(10) Pub. No.: US 2016/0158267 A1
(43) Pub. Date: Jun. 9, 2016

(54) METHODS OF PREVENTING, REDUCING OR TREATING MACULAR DEGENERATION

(71) Applicant: Inotek Pharmaceuticals Corporation, Lexington, MA (US)

(72) Inventor: William K. MCVICAR, Sudbury, MA (US)

(21) Appl. No.: 14/957,170

(22) Filed: Dec. 2, 2015

Related U.S. Application Data
(60) Provisional application No. 62/087,080, filed on Dec. 3, 2014.

(51) Int. Cl. A61K 31/7076; A61K 9/00

(52) U.S. Cl. CPC A61K 31/7076; A61K 9/0048

(57) ABSTRACT

The present invention is directed to selective adenosine A₁ agonist compounds, pharmaceutical compositions comprising such compounds, and methods of using such compounds to treat, reduce or prevent age-related macular degeneration.

### CROSS REFERENCE TO RELATED APPLICATIONS

This application claims priority to, and the benefit of, U.S. Application No. 62/087,080, filed on Dec. 3, 2014. The contents of the aforementioned application are hereby incorporated herein by reference.

### TECHNICAL FIELD OF THE INVENTION

Provided herein are methods of preventing or treating macular degeneration, in particular age-related macular degeneration, in one or more subjects in need thereof. More particularly, provided herein are methods of preventing or treating dry age-related macular degeneration. Also provided herein are uses of certain compounds in subjects for preventing, reducing or treating retinal pigment epithelium damage and photoreceptor cells damage associated with age-related macular degeneration. Also provided herein are methods for preventing, reducing or treating retinal pigment epithelium damage and/or photoreceptor cells damage.

### BACKGROUND OF THE INVENTION

The retinal pigment epithelium (RPE) is interposed between the neural retina and the choroid and plays a central role in retinal physiology by forming the outer blood-retinal barrier and supporting the structural and physiological integrities of neighboring tissues. For example, the retinal pigment epithelium provides nutritional support for photoreceptor cells and is involved in the processing and transport of metabolic waste from the photoreceptors across Bruch's membrane to the choroid.

The buildup of drusen proximate the retinal pigment epithelium results from tiny yellow or white accumulations of extracellular material that build up between Bruch's membrane (a membrane of the choroid, a network of blood vessels which supplies the retina with blood) and the retinal pigment epithelium of the eye. The presence of a few small (“hard”) drusen is normal with advancing age, and most people over 40 have some hard drusen. However, the presence of larger and more numerous drusen is a common early sign of age-related macular degeneration (AMD).

AMD begins with characteristic yellow deposits (drusen) in the macula, between the retinal pigment epithelium and the underlying choroid. Most people with these early changes (referred to as age-related maculopathy) still have good vision. People with drusen may or may not develop AMD, in fact the majority of people over age 55 have drusen with no negative effects. The risk of developing symptoms is higher when the drusen are large and numerous and associated with disturbance in the pigmented cell layer under the macula. Large and soft drusen are thought to be related to elevated cholesterol deposits.

There are two types of AMD. One is known as dry-AMD and the other is known as wet-AMD.

The “wet” or exudative form of AMD, which is more severe, occurs when blood vessels grow up from the choroid behind the retina, and the retina can become detached. Wet AMD can be treated with laser coagulation, and with medication that stops and sometimes reverses the growth of blood vessels.

The “dry” form of AMD, results from atrophy of the retinal pigment epithelial layer through the loss of RPE cells, which in advanced stages causes vision loss through loss of photoreceptors (rods and cones) which are supported by the retinal pigmented epithelium in the central part of the eye in a patient.

The dry (atrophic) type of AMD affects approximately 80-90% of subjects with AMD. Its cause is unknown, it tends to progress more slowly than the wet type, and there is not, as of yet, an approved treatment or cure.

There is, therefore, a need for other therapeutic agents that can (i) prevent macular degeneration, and/or (ii) stop or slow the progression of macular degeneration and/or (iii) treat or reverse macular degeneration, particularly dry age-related macular degeneration.

### SUMMARY OF THE INVENTION

Provided herein are selective adenosine A₁ agonist compounds, pharmaceutical compositions comprising such compounds, and methods of using such compounds to treat, reduce or prevent age-related macular degeneration.

Thus in a first aspect there is provided a method of preventing age-related macular degeneration in a subject comprising applying an effective amount of an ophthalmic pharmaceutical composition comprising a selective adenosine A₁ agonist to an eye of the subject.

In a second aspect, the present invention provides a method of reducing age-related macular degeneration in a subject by administering an effective amount of an ophthalmic pharmaceutical composition comprising a selective A₁ agonist to an affected eye of the subject.

In a third aspect, the present invention provides a method of treating a subject in need thereof from age-related macular degeneration, comprising the step of: applying a pharmaceutical composition comprising an effective amount of a selective A₁ agonist to an eye of the subject.

In a fourth aspect, the present invention provides a method for preventing, reducing or treating retinal pigment epithelium damage in a subject by administering an effective amount of an ophthalmic pharmaceutical composition comprising a selective A₁ agonist to an affected eye of the subject.

In a fifth aspect, the present invention provides a method for preventing, reducing or treating photoreceptor cell damage in a subject by administering an effective amount of an ophthalmic pharmaceutical composition comprising a selective A₁ agonist to an affected eye of the subject.

In some embodiments, the methods of the invention prevent or reduce atrophy of the retinal pigmented epithelium (RPE). In some embodiments, the methods of the invention prevent or reduce the loss of photoreceptor cells.

In one embodiment the age-related macular degeneration is dry-age related macular degeneration.

In certain embodiments, the methods comprise applying an effective amount of a selective adenosine A₁ agonist compound according to Formula I,

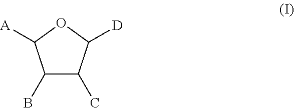

or a pharmaceutically acceptable salt thereof,
   wherein

A is —CH₂ONO₂, —CH₂OH, or —CH₂OSO₃H;

B and C are —OH; and

D is

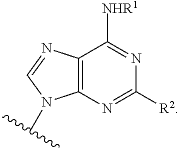

In certain embodiments of the methods of the invention, the adenosine A₁ agonist is Compound A:

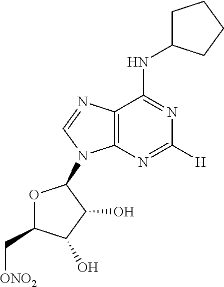

((2R,3S,4R,5R)-5-(6-(cyclopentylamino)-9H-purin-9-yl)-3,4-dihydroxytetrahydrofuran-2-yl)methyl nitrate.

The methods of the invention are useful for preventing, reducing or treating age-related macular degeneration in subjects having or at risk of developing dry age-related macular degeneration. In some embodiments, the methods of the invention are useful for preventing or reducing retinal epithelial cell damage and/or loss of photoreceptor cells in subjects having or at risk of developing age-related macular degeneration.

In one embodiment the diseases or conditions giving rise to age-related macular degeneration is not caused solely by elevated intraocular pressure.

When practicing the methods of the invention, the selective adenosine A₁ agonist can be administered in drops, e.g., 1 to 2 drops.

In some embodiments, of the methods described herein, the effective amount of the selective adenosine A₁ agonist applied to the eye is about 20 μg to about 7.0 mg. In some embodiments, the effective amount of the selective adenosine A₁ agonist is from about 30 μg to 1 mg. In some embodiments the effective amount of selective adenosine A₁ agonist is at least 20 μg. In some embodiments, the effective amount of the selective adenosine A₁ agonist is between 60 μg and 1500 μg; is about 100 μg, about 200 μg, about 250 μg, about 300 μg, about 350 μg, about 400 μg, about 450 μg, about 500 μg, about 550 μg or about 600 μg or about 500 to 1500 μg. In certain embodiments, the effective amount of the selective adenosine A₁ agonist is about 500 μg.

In one embodiment the selective adenosine A₁ agonist is administered at an effective dose of about 0.1 to about 5.0% (w/v). In one embodiment, the selective adenosine A₁ agonist is administered at an effective dose of about 0.5 to about 1.5% (w/v). In one embodiment, the selective adenosine A₁ agonist is administered at an effective dose of about 1.0% to about 3.0% (w/v). In one embodiment, selective adenosine A₁ agonist is administered at an effective dose of about 3.0% (w/v).

In one embodiment the effective amount of the selective adenosine A₁ agonist is administered as a single dose. In another embodiment, the effective amount of the selective adenosine A₁ agonist is administered as a twice daily dose. In another embodiment, the selective adenosine A₁ agonist is administered 1 to 4 times daily.

In certain embodiments, the selective adenosine A₁ agonist administered is selected from the group consisting of:
((2R,3S,4R,5R)-5-(6-(cyclopentylamino)-9H-purin-9-yl)-3,4-dihydroxytetrahydrofuran-2-yl)methyl nitrate;
((2R,3S,4R,5R)-5-(2-chloro-6-(cyclopentylamino)-9H-purin-9-yl)-3,4-dihydroxytetrahydrofuran-2-yl)methyl nitrate;
sodium ((2R,3S,4R,5R)-5-(6-(cyclopentylamino)-9H-purin-9-yl)-3,4-dihydroxytetrahydrofuran-2-yl)methyl sulfate;
((2R,3S,4R,5R)-3,4-dihydroxy-5-(6-(tetrahydrofuran-3-ylamino)-9H-purin-9-yl)tetrahydrofuran-2-yl)methyl nitrate;
((2R,3S,4R,5R)-5-(6-(cyclohexylamino)-9H-purin-9-yl)-3,4-dihydroxytetrahydrofuran-2-yl)methyl nitrate;
((2R,3S,4R,5R)-5-(6-(bicycle-[2.2.1]-heptan-2-ylamino)-9H-purin-9-yl)-3,4-dihydroxytetrahydrofuran-2-yl)methyl nitrate;
sodium ((2R,3S,4R,5R)-5-(2-chloro-6-(cyclohexylamino)-9H-purin-9-yl)-3,4-dihydroxytetrahydrofuran-2-yl)methyl sulfate;
((2R,3S,4R,5R)-5-(2-chloro-6-(cyclohexylamino)-9H-purin-9-yl)-3,4-dihydroxytetrahydrofuran-2-yl)methyl nitrate; cyclohexyladenosine (CHA) and 2-chlorocyclopentyladenosine (CCPA) and cyclopentyladenosine (CPA).

In one embodiment the compound administered is selected from: ((2R,3S,4R,5R)-5-(2-chloro-6-(cyclopentylamino)-9H-purin-9-yl)-3,4-dihydroxytetrahydrofuran-2-yl)methyl nitrate;

and
((2R,3S,4R,5R)-3,4-dihydroxy-5-(6-(tetrahydrofuran-3-ylamino)-9H-purin-9-yl)tetrahydrofuran-2-yl)methyl nitrate.

It is to be further appreciated that the compounds of Formula I as defined above, may be used in the manufacture of a medicament for preventing, reducing or treating age-related macular degeneration in an affected eye of a subject.

The foregoing brief summary broadly describes the features and technical advantages of certain embodiments of the present invention. Further technical advantages will be described in the detailed description of the invention that follows. Novel features which are believed to be characteristic of the invention will be better understood from the detailed description of the invention when considered in connection with any accompanying figures and examples. However, the figures and examples provided herein are intended to help illustrate the invention or assist with developing an understanding of the invention, and are not intended to be definitions of the invention's scope.

### BRIEF DESCRIPTION OF THE DRAWINGS

FIG. 1 shows a retinal cross section of a rat retina. P indicates the photoreceptor layer; O indicates the outer nuclear layer and RPE indicates the Retinal Pigmented Epithelium. HE, 400x.

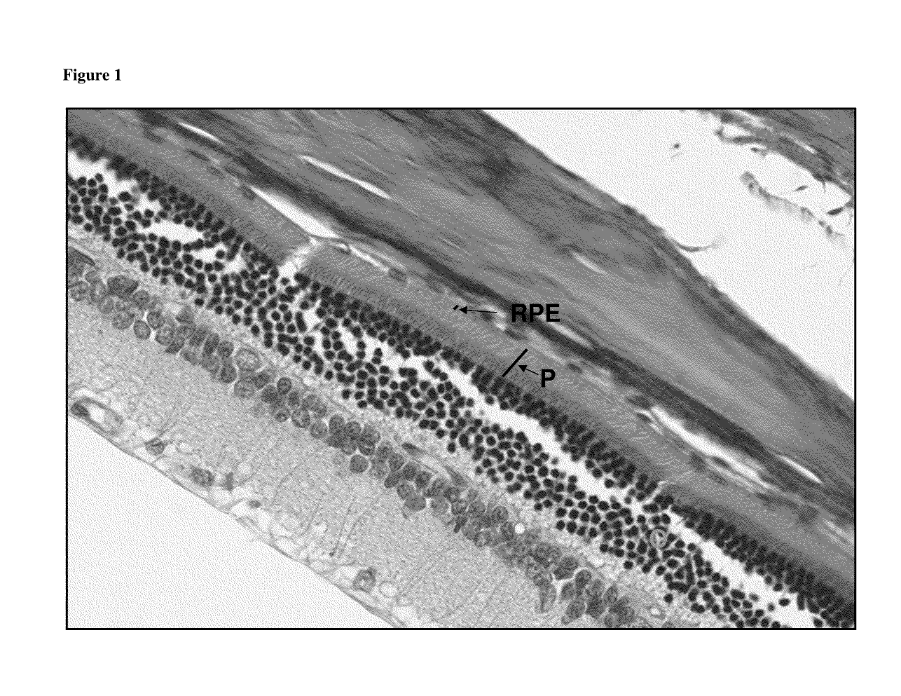

FIG. 2 shows a summary plot of Electroretinography—Scotopic Single Flash 0 dB—A-Wave—Males for Example 1

FIG. 3 shows a summary plot of Electroretinography—Scotopic Single Flash 0 dB—B-Wave—Males for Example 1

FIG. 4 shows a summary plot of Electroretinography—Scotopic Flash Stimuli 0 dB—B-Wave for Example 2

FIG. 5 shows a plot of the outer nuclear layer (ONL) thickness in terms of number of cells across the various treatment groups for Example 2

### DETAILED DESCRIPTION OF THE INVENTION

Embodiments of the present invention provide compounds useful for preventing, reducing or treating age related macular degeneration, in particular dry age related macular degeneration. For example, use of the compounds provided herein are useful for preventing or reducing the loss of RPE cells and photoreceptors, associated with age related decline in RPE function.

During development, the retina forms as an outpocketing of the diencephalon, called the optic vesicle, which undergoes invagination to form the optic cup. The inner wall of the optic cup gives rise to the retina, while the outer wall gives rise to the retinal pigment epithelium a melanin-containing structure that reduces backscattering of light that enters the eye, which also plays a critical role in the maintenance of photoreceptors, renewing photopigments and phagocytosing the photoreceptor disks, whose turnover at a high rate is essential to vision. The retina also comprises complex neural circuitry in which a three-neuron chain, photoreceptor cell to bipolar cell to ganglion cell, is the major route of information from photoreceptors to the optic nerve.

Adenosine is a purine nucleoside that modulates many physiologic processes. Cellular signaling by adenosine occurs through four adenosine receptor subtypes: A₁, A_{2A}, A_{2B}, and A₃ as reported by Ralevic and Burnstock (Pharmacol Rev. 50:413-492, 1988) and Fredholm BB et al. (Pharmacol Rev. 53:527-552, 2001). The presence of A₁ receptor subtypes on retinal pigment epithelium was reported by Collison DJ et al (Exp Eye Res Apr 80(4): 465-75, 2005). Acute and chronic animal models of optic nerve degeneration have shown the neuroprotective potential of the alpha2 adrenergic agonist brimonidine. These models include direct injury of the optic nerve (nerve crush) and models of acute and chronic ocular hypertension (Yoles et al 1999; Donello et al 2001; WoldeMussie et al 2001; Mayor-Torroglosa et al 2005; Lambert et al 2011). In a clinical study of patients with low pressure glaucoma, 0.2% brimonidine was also shown to slow the loss of visual field (Krupin et al. Am J Ophthalmol. 2011; 151:671-681).

Compounds that act as selective adenosine A₁ agonists are known and have shown a variety of utilities. Selective adenosine A₁ agonists have been discovered to reduce IOP in humans in clinical studies as published in PCT/US2010/033112.

In particular, described herein are compounds of Formula I (e.g., Compounds A, B, C, D, E, F, G, H, I, J or K) that can prevent, treat or reduce aged macular degeneration in a subject (e.g., a human) in need thereof.

Compounds of Formula I have the following structure:

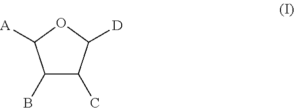

or a pharmaceutically acceptable salt thereof,
   wherein

A is —CH₂ONO₂, —CH₂OH, or —CH₂OSO₃H;

B and C are —OH;

D is

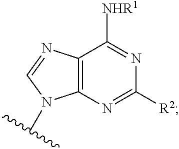

A and B are trans with respect to each other;

B and C are cis with respect to each other;

C and D are cis or trans with respect to each other;

R¹ is —H, —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;

R² is —H, halo, —CN, —NHR⁴, —NHC(O)R⁴, —NHC(O)OR⁴, —NHC(O)NHR⁴, —NHNHC(O)R⁴, —NHNHC(O)OR⁴, —NHNHC(O)NHR⁴, or —NH—N═C(R⁶)R⁷;

R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;

R⁶ is —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ—(C₃-C₈ monocyclic cycloalkenyl), -phenylene-(CH₂)ₙCOOH, or -phenylene-(CH₂)ₙCOO—(C₁-C₁₀ alkyl);

R⁷ is —H, —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkenyl) or —(CH₂)ₙ—(C₈-C₁₂ bicyclic cycloalkyl); and

each n is independently an integer ranging from 1 to 5, and a pharmaceutically acceptable vehicle.

In a further embodiment, the compounds for use in the invention are compounds having the formula

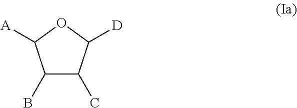

or a pharmaceutically acceptable salt thereof,
   wherein

A is —CH₂ONO₂, —CH₂OH, or —CH₂OSO₃H;

B and C are —OH;

D is

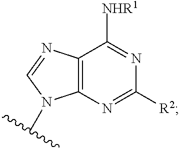

A and B are trans with respect to each other;

B and C are cis with respect to each other;

C and D are cis or trans with respect to each other;

R¹ is —C₃-C₈ monocyclic cycloalkyl, -3- to 7-membered monocyclic heterocycle, or —C₈-C₁₂ bicyclic cycloalkyl; and

R² is —H or -halo.

In a further embodiment, the compounds for use in the invention are compounds having the formula

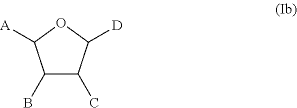

or a pharmaceutically acceptable salt thereof,
   wherein

A is —CH₂ONO₂;

B and C are —OH;

D is

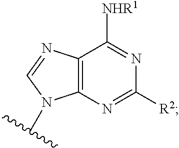

A and B are trans with respect to each other;

B and C are cis with respect to each other;

C and D are cis or trans with respect to each other;

R¹ is —C₃-C₈ monocyclic cycloalkyl, -3- to 7-membered monocyclic heterocycle, or —C₈-C₁₂ bicyclic cycloalkyl; and

R² is —H or -halo.

In another embodiment, the compound of Formula I is one of the following compounds:

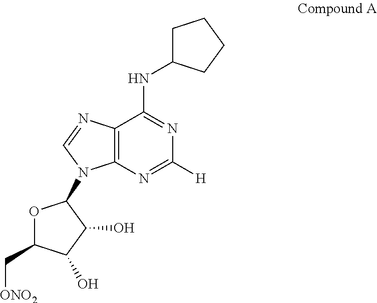

((2R,3S,4R,5R)-5-(6-(cyclopentylamino)-9H-purin-9-yl)-3,4-dihydroxytetrahydrofuran-2-yl)methyl nitrate,

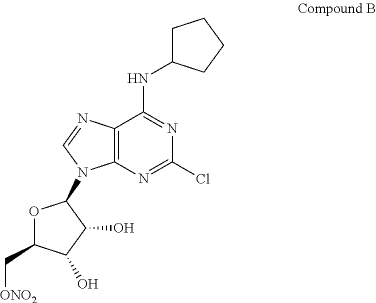

((2R,3S,4R,5R)-5-(2-chloro-6-(cyclopentylamino)-9H-purin-9-yl)-3,4-dihydroxytetrahydrofuran-2-yl)methyl nitrate,

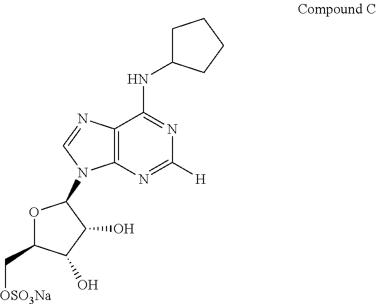

sodium ((2R,3S,4R,5R)-5-(6-(cyclopentylamino)-9H-purin-9-yl)-3,4-dihydroxytetrahydrofuran-2-yl)methyl sulfate,

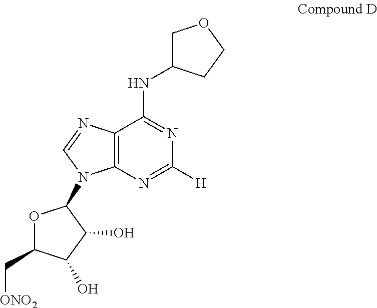

((2R,3S,4R,5R)-3,4-dihydroxy-5-(6-(tetrahydrofuran-3-ylamino)-9H-purin-9-yl)tetrahydrofuran-2-yl)methyl nitrate,

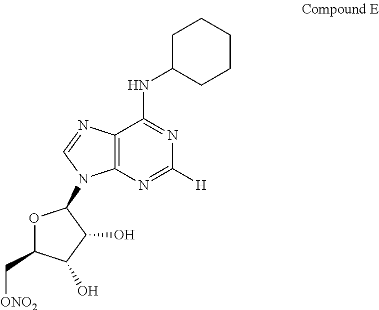

((2R,3S,4R,5R)-5-(6-(cyclohexylamino)-9H-purin-9-yl)-3,4-dihydroxytetrahydrofuran-2-yl)methyl nitrate,

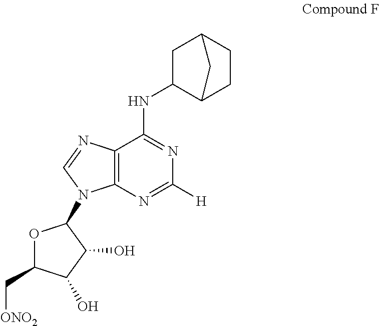

((2R,3S,4R,5R)-5-(6-(bicycle-[2.2.1]-heptan-2-ylamino)-9H-purin-9-yl)-3,4-dihydroxytetrahydrofuran-2-yl)methyl nitrate,

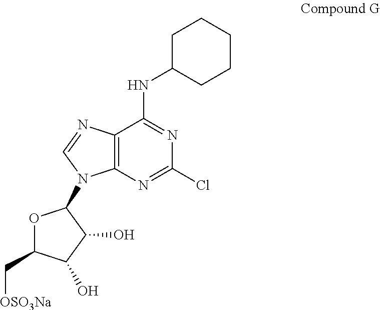

sodium ((2R,3S,4R,5R)-5-(2-chloro-6-(cyclohexylamino)-9H-purin-9-yl)-3,4-dihydroxytetrahydrofuran-2-yl)methyl sulfate,

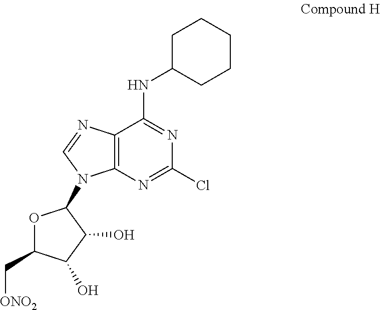

((2R,3S,4R,5R)-5-(2-chloro-6-(cyclohexylamino)-9H-purin-9-yl)-3,4-dihydroxytetrahydrofuran-2-yl)methyl nitrate, and

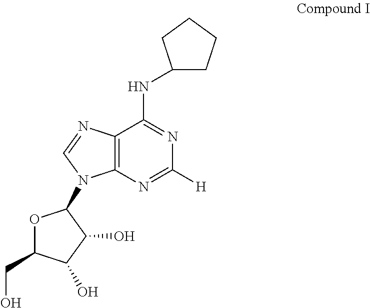 [SMILES: OC[C@H]1O[C@@H](n2cnc3c(NC4CCCC4)ncnc32)[C@H](O)[C@@H]1O]

Cyclopentyladensine (CPA),

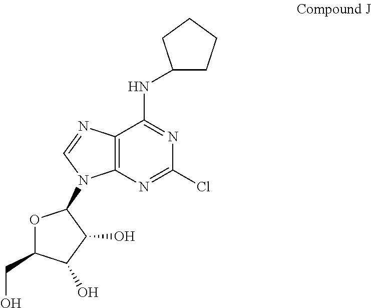 [SMILES: OC[C@H]1O[C@@H](n2cnc3c(NC4CCCC4)nc(Cl)nc32)[C@H](O)[C@@H]1O]

2-chlorocyclopentyladenosine (CCPA),

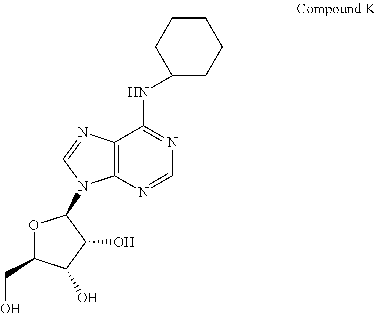 [SMILES: OC[C@H]1O[C@@H](n2cnc3c(NC4CCCCC4)ncnc32)[C@H](O)[C@@H]1O]

Cyclohexyladenosine (CHA), or pharmaceutically acceptable salts thereof.

Where discrepancies exist between a compound's name and a compound's structure, the chemical structure will control.

One of the most common early signs of age related macular degeneration is the presence of drusen, tiny yellow deposits under the retina or pigment clumping. These may be detected during a routine eye exam (e.g., dilation), and can be seen by Fundus photography. Other tests used to detect the presence of age related macular degeneration include Amsler grid, Snellen chart and contrast sensitivity tests (e.g., to measure the ability to see contours, shadows and color), optical coherence tomography, and structured illumination light microscopy. For wet macular degeneration, angiography (e.g., fluorescein angiography) allows the identification and localization of abnormal vascular processes including leakage of blood vessels behind the macula.

There are a number of methods which can be used to measure the function of photoreceptor cells. For example, damage to photoreceptor cells can be measured using electroretinogram (ERG) or electroretinography measurements. Electroretinography measures electrical activity generated by the photoreceptor cells in the retina when the eye is stimulated by certain light sources. The measurement is captured by electrodes placed on the front surface of the eye (e.g. cornea) and the skin near the eye and a graphic record called an electroretinogram (ERG) is produced. Electroretinography is useful in diagnosing several hereditary and acquired disorders of the retina, such as but not limited retinitis pigmentosa, a detached retina or functional changes caused by arteriosclerosis or diabetes.

In one embodiment, provided herein is a method of preventing age related macular degeneration, comprising administering an effective amount of a compound of Formula I to an eye of a subject.

In another embodiment, provided herein is a method of reducing or treating age related macular degeneration, comprising applying an effective amount of a compound of Formula I to an affected eye of a subject.

In another embodiment, provided herein is a method of preventing, reducing or treating age related macular degeneration, comprising applying an effective amount of a compound of Formula I to an eye of a subject. In another embodiment, about 0.1 to 3.0% (w/v) of a compound of Formula I is applied to an eye of a subject from 1 to 4 times daily. In one embodiment, about 0.5 to about 1.5% (w/v) of a compound of Formula I is applied to an eye of a human from 1 to 4 times daily. In still another embodiment, about 1.5% (w/v) of a compound of Formula I is applied to an eye of a human from 1 to 4 times daily. In one embodiment, the compound of Formula I is applied twice daily. In one embodiment, the compound of Formula I is applied once daily. A compound of Formula I can be administered in drops, e.g., 1 to 2 drops.

In another embodiment, provided herein is a method of preventing, reducing or treating age related macular degeneration, comprising administering an effective amount of Compound A to a subject. In still another embodiment, provided herein is a method of preventing, reducing or treating age related macular degeneration, comprising applying an effective amount of Compound A to an eye of a subject. In one embodiment, about 0.5 to about 1.5% (w/v) of Compound A is applied to an eye of a subject from 1 to 4 times daily. In another embodiment, about 0.5 to about 1.5% (w/v) of Compound A is applied to an eye of a subject from 1 to 4 times daily. In another embodiment, about 1.5% (w/v) of Compound A is applied to an eye of a subject from 1 to 4 times daily. In one embodiment, the compound of Formula I is applied twice daily. In one embodiment, the compound of Formula I is applied once daily. The Compound A can be administered in drops, e.g., 1 to 2 drops.

In another embodiment, provided herein is the use of a compound of Formula I for the manufacture of a medicament for preventing, reducing or treating age related macular degeneration in a subject. In another embodiment, provided herein is the use of a compound of Formula I for the manufacture of a medicament for reducing age related macular degeneration in a subject. In another embodiment, provided herein is the use of a compound of Formula I for the manufacture of a medicament for treating age related macular degeneration in a subject.

In another embodiment, provided herein is the use of a compound of Formula I for preventing age related macular degeneration in a subject.

In another embodiment, provided herein is the use of a compound of Formula I for reducing or treating age related macular degeneration in a subject.

In another embodiment, provided herein is the use of Compound A for preventing age related macular degeneration in a subject. In another embodiment, provided herein is the use of Compound A for reducing age related macular degeneration in a subject. In another embodiment, provided herein is the use of Compound A for treating age related macular degeneration in a subject.

It is recognized that compounds of Formula I can contain one or more chiral centers. This invention contemplates all enantiomers, diastereomers, and mixtures of Formulas I thereof.

Furthermore, certain embodiments of the present invention comprise pharmaceutically acceptable salts of compounds according to Formula I.

Pharmaceutically acceptable salts comprise, but are not limited to, soluble or dispersible forms of compounds according to Formula I that are suitable for treatment of disease without undue undesirable effects such as allergic reactions or toxicity.

Representative pharmaceutically acceptable salts include, but are not limited to, acid addition salts such as acetate, citrate, benzoate, lactate, or phosphate and basic addition salts such as lithium, sodium, potassium, or aluminum.

### Definitions

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

As used herein, the term “selective adenosine A₁ agonist” means an A₁ agonist that has a high affinity to the A₁ receptor while simultaneously having a lower affinity for the A_{2A,} and A₃ adenosine receptors. Compounds of Formula I (e.g., Compounds A to K) above have affinities to the A₁ receptor considerably greater than their respective affinities to the A_{2A} and A₃ receptors. The A₁ selectivity data for compounds A to K is summarized in the Table below.

| | | A₁ > A_{2A} | A₁ > A₃ |
|---|---|---|---|
| | | SELECTIVITY | SELECTIVITY |
| | A₁ (Ki (nm)) | [KiA₂(nm)/ | [KiA₃(nm)/ |
| Compound | POTENCY | KiA₁(nm)] | KiA₁(nm)] |
| Compound A | 0.97 | 4837 | 725 |
| Compound B | 2.63 | 1593 | 195 |
| Compound C | 4.05 | 2250 | 251 |
| Compound D | 10.6 | >9434 | 202 |
| Compound E | 1.32 | 878 | 1098 |
| Compound F | 1.47 | 3945 | 260 |
| Compound G | 1.36 | 200 | 130 |
| Compound H | 8 | 192 | 167 |
| Compound I | 2.3 | 345 | 31.3 |
| (CPA) | | | |
| Compound J | 0.83 | 2735 | 50 |
| (CCPA) | | | |
| Compound K | 0.732 | 839 | 206 |
| (CHA) | | | |

As used herein, the term “alkyl” refers to a fully saturated branched or unbranched hydrocarbon moiety. Preferably the alkyl comprises 1 to 20 carbon atoms, more preferably 1 to 16 carbon atoms, 1 to 10 carbon atoms, 1 to 7 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like. Furthermore, the expression “Cₓ-C_{y}-alkyl”, wherein x is 1-5 and y is 2-15 indicates a particular alkyl group (straight- or branched-chain) of a particular range of carbons. For example, the expression C₁-C₄-alkyl includes, but is not limited to, methyl, ethyl, propyl, butyl, isopropyl, tert-butyl and isobutyl. The term alkyl includes, but is not limited to, C₁-C₁₅ alkyl, C₁-C₁₀ alkyl and C₁-C₆ alkyl.

The term “C₁-C₁₅ alkyl” as used herein refers to a straight or branched chain, saturated hydrocarbon having from 1 to 15 carbon atoms. Representative C₁-C₁₅ alkyl groups include, but are not limited to methyl, ethyl, propyl, isopropyl, butyl, sec-butyl, tert-buty, pentyl, isopentyl, neopentyl, hexyl, isohexyl, neohexyl, heptyl, isoheptyl, neoheptyl, octyl, isooctyl, neooctyl, nonyl, isononyl, neononyl, decyl, isodecyl, neodecyl, undecyl, dodecyl, tridecyl, tetradecyl and pentadecyl. In one embodiment, the C₁-C₁₅ alkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₁-C₁₅ alkyl is unsubstituted.

The term “C₁-C₁₀ alkyl” as used herein refers to a straight or branched chain, saturated hydrocarbon having from 1 to 10 carbon atoms. Representative C₁-C₁₀ alkyl groups include, but are not limited to methyl, ethyl, propyl, isopropyl, butyl, sec-butyl, tert-butyl, pentyl, isopentyl, neopentyl, hexyl, isohexyl, neohexyl, heptyl, isoheptyl, neoheptyl, octyl, isooctyl, neooctyl, nonyl, isononyl, neononyl, decyl, isodecyl and neodecyl. In one embodiment, the C₁-C₁₀ alkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₁-C₁₀ alkyl is unsubstituted. C₁-C₁₀ alkyl includes, but is not limited to, C₁-C₆ alkyl.

The term “C₁-C₆ alkyl” as used herein refers to a straight or branched chain; saturated hydrocarbon having from 1 to 6 carbon atoms. Representative C₁-C₆ alkyl groups include, but are not limited to methyl, ethyl, propyl, isopropyl, butyl, sec-butyl, tert-buty, pentyl, isopentyl, neopentyl, hexyl, isohexyl, and neohexyl. Unless indicated, the C1-C6 alkyl is unsubstituted.

The term “aryl” as used herein refers to a phenyl group or a naphthyl group. In one embodiment, the aryl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the aryl is unsubstituted.

The term “C₃-C₈ monocyclic cycloalkyl” as used herein is a 3-, 4-, 5-, 6-, 7- or 8-membered saturated non-aromatic monocyclic cycloalkyl ring. Representative C₃-C₈ monocyclic cycloalkyl groups include, but are not limited to, cyclopropyl, cyclobutyl, cyclopentyl, cyclohexyl, cycloheptyl and cyclooctyl. In one embodiment, the C₃-C₈ monocyclic cycloalkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₃-C₈ monocyclic cycloalkyl is unsubstituted.

The term “C₃-C₈ monocyclic cycloalkenyl” as used herein is a 3-, 4-, 5-, 6-, 7- or 8-membered non-aromatic monocyclic carbocyclic ring having at least one endocyclic double bond, but which is not aromatic. It is to be understood that when any two groups, together with the carbon atom to which they are attached form a C₃-C₈ monocyclic cycloalkenyl group, the carbon atom to which the two groups are attached remains tetravalent. Representative C₃-C₈ monocyclic cycloalkenyl groups include, but are not limited to, cyclopropenyl, cyclobutenyl, 1,3-cyclobutadienyl, cyclopentenyl, 1,3-cyclopentadienyl, cyclohexenyl, 1,3-cyclohexadienyl, cycloheptenyl, 1,3-cycloheptadienyl, 1,4-cycloheptadienyl, -1,3,5-cycloheptatrienyl, cyclooctenyl, 1,3-cyclooctadienyl, 1,4-cyclooctadienyl, -1,3,5-cyclooctatrienyl. In one embodiment, the C₃-C₈ monocyclic cycloalkenyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₃-C₈ monocyclic cycloalkenyl is unsubstituted.

The term “C₈-C₁₂ bicyclic cycloalkyl” as used herein is a 8-, 9-, 10-, 11- or 12-membered saturated, non-aromatic bicyclic cycloalkyl ring system. Representative C₈-C₁₂ bicyclic cycloalkyl groups include, but are not limited to, decahydronaphthalene, octahydroindene, decahydrobenzocycloheptene, and dodecahydroheptalene. In one embodiment, the C₈-C₁₂ bicyclic cycloalkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₈-C₁₂ bicyclic cycloalkyl is unsubstituted.

The term “C₈-C₁₂ bicyclic cycloalkenyl” as used herein is a 8-, 9-, 10-, 11- or 12-membered non-aromatic bicyclic cycloalkyl ring system, having at least one endocyclic double bond. It is to be understood that when any two groups, together with the carbon atom to which they are attached form a C₈-C₁₂ bicyclic cycloalkenyl group, the carbon atom to which the two groups are attached remains tetravalent. Representative C₈-C₁₂ bicyclic cycloalkenyl groups include, but are not limited to, octahydronaphthalene, hexahydronaphthalene, hexahydroindene, tetrahydroindene, octahydrobenzocycloheptene, hexahydrobenzocycloheptene, tetrahydrobenzocyclopheptene, decahydroheptalene, octahydroheptalene, hexahydroheptalene, and tetrahydroheptalene. In one embodiment, the C₈-C₁₂ bicyclic cycloalkyl group is substituted with one or more of the following groups: -halo, -0-(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₈-C₁₂ bicyclic cycloalkenyl is unsubstituted.

The term “halo” as used herein refers to —F, —Cl, —Br or —I.

The term “3- to 7-membered monocyclic heterocycle” refers to: (i) a 3- or 4-membered non-aromatic monocyclic cycloalkyl in which 1 of the ring carbon atoms has been replaced with an N, O or S atom; or (ii) a 5-, 6-, or 7-membered aromatic or non-aromatic monocyclic cycloalkyl in which 1-4 of the ring carbon atoms have been independently replaced with a N, O or S atom. The non-aromatic 3- to 7-membered monocyclic heterocycles can be attached via a ring nitrogen, sulfur, or carbon atom. The aromatic 3- to 7-membered monocyclic heterocycles are attached via a ring carbon atom. Representative examples of a 3- to 7-membered monocyclic heterocycle group include, but are not limited to furanyl, furazanyl, imidazolidinyl, imidazolinyl, imidazolyl, isothiazolyl, isoxazolyl, morpholinyl, oxadiazolyl, oxazolidinyl, oxazolyl, oxazolidinyl, pyrimidinyl, phenanthridinyl, phenanthrolinyl, piperazinyl, piperidinyl, pyranyl, pyrazinyl, pyrazolidinyl, pyrazolinyl, pyrazolyl, pyridazinyl, pyridooxazole, pyridoimidazole, pyridothiazole, pyridinyl, pyrimidinyl, pyrrolidinyl, pyrrolinyl, quinuclidinyl, tetrahydrofuranyl, thiadiazinyl, thiadiazolyl, thienyl, thienothiazolyl, thienooxazolyl, thienoimidazolyl, thiomorpholinyl, thiophenyl, triazinyl, triazolyl, In one embodiment, the 3- to 7-membered monocyclic heterocycle group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the 3- to 7-membered monocyclic heterocycle is unsubstituted.

The term “8- to 12-membered bicyclic heterocycle” refers to a bicyclic 8- to 12-membered aromatic or non-aromatic bicyclic cycloalkyl in which one or both of the of the rings of the bicyclic ring system have 1-4 of its ring carbon atoms independently replaced with a N, O or S atom. Included in this class are 3- to 7-membered monocyclic heterocycles that are fused to a benzene ring. A non-aromatic ring of an 8- to 12-membered monocyclic heterocycle is attached via a ring nitrogen, sulfur, or carbon atom. An aromatic 8- to 12-membered monocyclic heterocycles are attached via a ring carbon atom. Examples of 8- to 12-membered bicyclic heterocycles include, but are not limited to, benzimidazolyl, benzofuranyl, benzothiofuranyl, benzothiophenyl, benzoxazolyl, benzthiazolyl, benztriazolyl, benztetrzolyl, benzisoxazolyl, benzisothiazolyl, benzimidazolinyl, cinnolinyl, decahydroquinolinyl, 1H-indazolyl, indolenyl, indolinyl, indolizinyl, indolyl, isobenzofuranyl, isoindazolyl, isoindolyl, isoindolinyl, isoquinolinyl, naphthyridinyl, octahydroisoquinolinyl, phthalazinyl, pteridinyl, purinyl, quinoxalinyl, tetrahydroisoquinolinyl, tetrahydroquinolinyl, and xanthenyl. In one embodiment, each ring of a the -8- to 12-membered bicyclic heterocycle group can substituted with one or more of the following groups: -halo, —O—(C₁-C₆ alkyl), —OH, —CN, —COOR′, —OC(O)R′, —N(R′)₂, —NHC(O)R′. or —C(O)NHR′ groups wherein each R′ is independently —H or unsubstituted —C₁-C₆ alkyl. Unless indicated, the 8- to 12-membered bicyclic heterocycle is unsubstituted. Representative examples of a “phenylene group” are depicted below:

The phrase “pharmaceutically acceptable salt,” as used herein, is a salt of an acid and a basic nitrogen atom of a purine compound. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The pharmaceutically acceptable salt can also be a camphorsulfonate salt. The term “pharmaceutically acceptable salt” also refers to a salt of a purine compound having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like. The term “pharmaceutically acceptable salt” also includes a hydrate of a purine compound.

Some chemical structures herein are depicted using bold and dashed lines to represent chemical bonds. These bold and dashed lines depict absolute stereochemistry. A bold line indicates that a substituent is above the plane of the carbon atom to which it is attached and a dashed line indicates that a substituent is below the plane of the carbon atom to which it is attached.

The term “effective amount” as used herein refers to an amount of a selective adenosine A₁ agonist that is effective for: (i) preventing age-related macular degeneration (ii) reducing or slowing the progression of age-related macular degeneration or (iii) treating age-related macular degeneration in a subject or (iv) preventing the loss or damage of RPE cells or the loss or damage of photoreceptors or (v) reducing or slowing the loss or damage of RPE cells or the loss or damage of photoreceptors or (vi) treating conditions or diseases caused by the loss or damage of RPE cells or the loss or damage of photoreceptors The term “subject” is intended to include organisms which are at risk of developing or are afflicted with a disease, disorder or condition associated with age-related macular degeneration. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of developing or potentially capable of suffering from age-related macular degeneration.

The term “treat,” “treated,” “treating” or “treatment” includes the diminishment or alleviation of at least one symptom associated or caused by the state, disorder or disease being treated. For example, the term “treat” may mean to reduce or prevent further damage or loss (e.g., of RPE and/or photoreceptors) such as that caused by, but not limited to age-related macular degeneration. For example, treatment can be diminishment of one or several symptoms of a disorder or complete eradication of a disorder. For example, symptoms of age-related macular degeneration include the presence of drusen, loss or damage of RPE cells, loss or damage of photoreceptors, loss of contrast sensitivity, blurred or blind spots in the center field of vision, and haziness of central or overall vision.

The terms “protect” or “prevent” are used interchangeably herein to delay the onset (i.e., the period prior to clinical manifestation of a disease) and/or to reduce the likelihood of a subject developing the disease (e.g., a subject at risk of developing a disease), or worsening of the disease (e.g., by stopping or slowing the progression of the disease in a subject with the disease). For example, the methods of the invention may be used to prevent, reduce or treat the loss or damage of RPE cells or the loss or damage of photoreceptors, such as that seen in, but not limited to age-related macular degeneration.

The term “use” includes any one or more of the following embodiments of the invention, respectively: the use in the treatment, prevention or reduction of the loss or damage of RPE cells, or the loss or damage of photoreceptors, such as that seen in, but not limited to age-related macular degeneration; the use for the manufacture of pharmaceutical compositions for use in the treatment of the diseases or conditions giving rise to the loss or damage of RPE cells, or the loss or damage of photoreceptors, such as that seen in, but not limited to age-related macular degeneration, e.g., in the manufacture of a medicament; methods of use of compounds of the invention in the treatment of such diseases or conditions; pharmaceutical preparations having compounds of the invention for the treatment of the loss or damage of RPE cells, loss or damage of photoreceptors, such as that seen in, but not limited to age-related macular degeneration; and compounds of the invention for use in the treatment of the loss or damage of RPE cells, loss or damage of photoreceptors, such as that seen in, but not limited to age-related macular degeneration; as appropriate and expedient, if not stated otherwise.

The term “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

As used herein, the term “drop” refers to a quantity of ophthalmically acceptable fluid that resembles a liquid drop. In one embodiment, a drop refers to a liquid volume equivalent to about 5 μl to about 200 μl, e.g., about 30 μl to about 80 μl.

The following abbreviations are used herein and have the indicated definitions: CCPA is 2-chloro-N6-cyclopentyladenosine; CPA is N6-cyclopentyladenosine; NECA is adenosine-5′-(N-ethyl)carboxamido; NMR is nuclear magnetic resonance; R-PIA is N6-(2-phenyl-isopropyl) adenosine, R-isomer; HPβCD is hydroxypropyl β-cyclodextrin.

### Methods of Synthesis

Compounds according to Formula I can be prepared by using synthetic procedures described in U.S. Pat. No. 7,423,144, the disclosure of which is incorporated herein in its entirety, as well as other published methods (see Cristalli et al., J. Med. Chem. 35:2363-2369, 1992; Cristalli et al., J. Med. Chem. 37:1720-1726, 1994; Cristalli et al, J. Med. Chem. 38:1462-1472, 1995; and Camaioni et al., Bioorg. Med. Chem. 5:2267-2275, 1997), or by using the synthetic procedures outlined below.

Scheme 1 shows methods for making nucleoside intermediates that are useful for making the compounds of the invention.

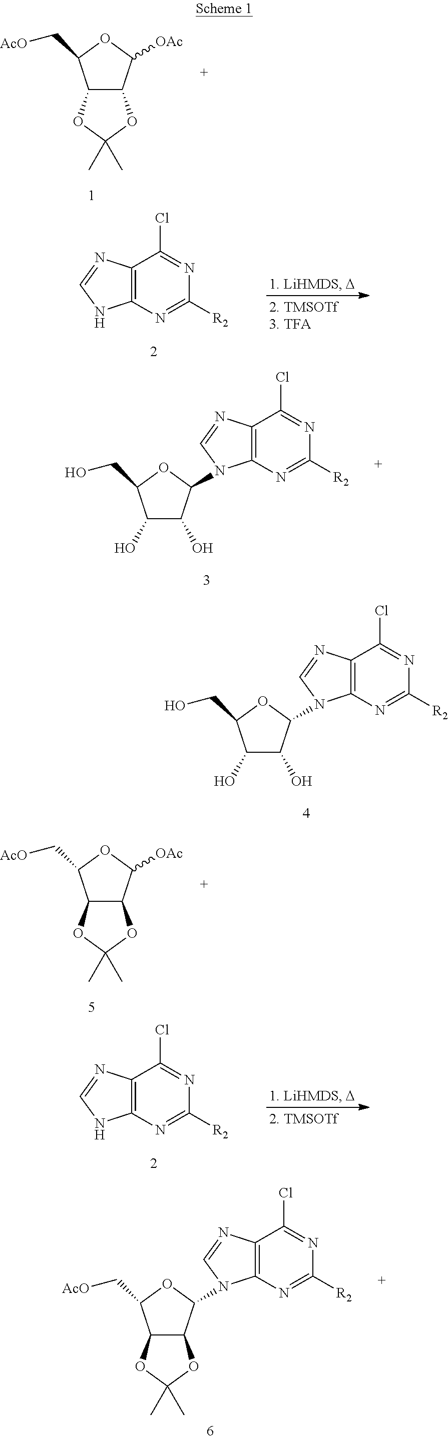

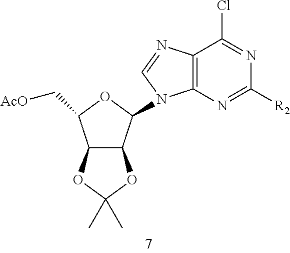

wherein R₂ is as defined above.

The protected ribose compound of Formula 1 can be coupled with a purine compound of Formula 2 using lithium hexamethyldisilazide and trimethylsilyl triflate, followed by acetonide removal using trifluoroacetic acid to provide nucleoside intermediates of Formula 3 and their corresponding other anomers of Formula 4. Similarly, the ribose diacetate of Formula 5 can be coupled with a compound of Formula 2 using lithium hexamethyldisilazide and trimethylsilyl triflate to provide acetonide-protected nucleoside intermediates of Formula 6 and their corresponding other anomers of Formula 7.

Scheme 2 shows a method useful for making the adenosine intermediates of Formula 8 which are useful for making the compounds of the invention.

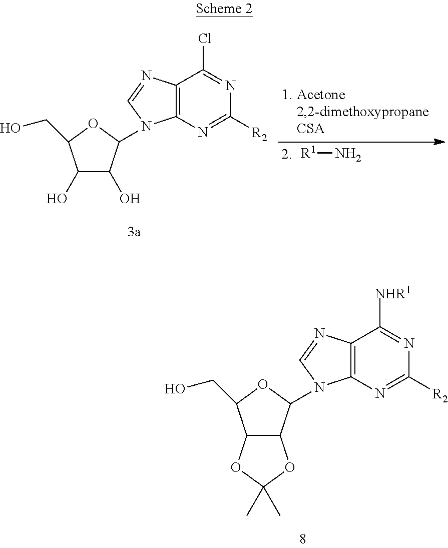

where R¹ and R² are defined above.

The 6-chloroadenosine derivative of formula 3a is converted to its 2′,3′-acetonide using acetone and 2,2-dimethoxypropane in the presence of camphorsulfonic acid. The acetonide can be further derivatized using an amine of formula R¹—NH₂ in the presence of base to provide compounds of formula 8.

Methodology useful for making other compounds of the invention is described in Scheme 4.

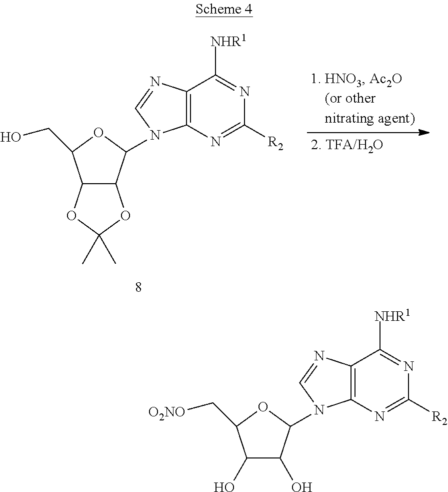

where R¹ and R² are defined above.

The adenosine intermediates of formula 8 can be converted to their 5′-nitrate analogs using nitric acid in the presence of acetic anhydride, or other nitrating agents, such as MsCl/ONO₃ or nitrosonium tetrafluoroborate. Acetonide removal using TFA/water provides compounds of the invention.

Methodology useful for making the Purine Derivatives of Formula (Id) wherein R³ is —CH₂OSO₃H is outlined in Scheme 6.

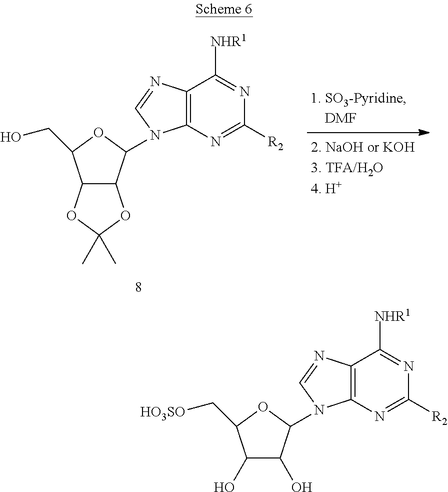

where R¹ and R² are defined above.

The adenosine intermediates of formula 8 can be treated with sulfur trioxide-pyridine complex to provide the corresponding 5′-sulfonic acid pyridine salt intermediate. The pyridine salt intermediate can then be neutralized using NaOH or KOH, followed by acetonide removal using TFA/water to provide the corresponding sodium or potassium salt, respectively, of the Purine Derivatives of Formula (Id) wherein A is —CH₂OSO₃H. Treatment of the sodium or potassium salt with strong aqueous acid, such as sulfuric or hydrochloric acid, provides compounds of the invention wherein A is —CH₂OSO₃H.

### Modes of Delivery

The compounds according to Formula I can be incorporated into various types of ophthalmic compositions or formulations for delivery. Formula I compounds may be delivered directly to the eye (for example: topical ocular drops or ointments; slow release devices such as pharmaceutical drug delivery sponges implanted in the cul-de-sac or implanted adjacent to the sclera or within the eye; periocular, conjunctival, sub-tenons, intracameral, intravitreal, or intracanalicular injections) or systemically (for example: orally, intravenous, subcutaneous or intramuscular injections; parenterally, dermal or nasal delivery) using techniques well known by those of ordinary skill in the art. It is further contemplated that the agents of the invention may be formulated in intraocular insert or implant devices.

The compounds of Formula I are preferably incorporated into topical ophthalmic formulations with a pH of about 4-8 for delivery to the eye. Various formulations of Compound A, in particular are described in PCT/US2010/033112, PCT/US2010/054040, and PCT/US2014/152723 entitled “Ophthalmic Formulations”, the contents of which are herein incorporated as if individually set forth.

The compounds may be combined with ophthalmologically acceptable preservatives, surfactants, viscosity enhancers, penetration enhancers, particle stabilizers, buffers, sodium chloride, and water to form an aqueous, sterile ophthalmic suspension or solution. Ophthalmic solution formulations may be prepared by dissolving a compound in a physiologically acceptable isotonic aqueous buffer. Further, the ophthalmic solution may include an ophthalmologically acceptable surfactant to assist in dissolving the compound. Furthermore, the ophthalmic solution may contain an agent to increase viscosity or solubility such as hydroxypropyl β-Cyclodextrin (HPβCD), hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient may be combined with a preservative in an appropriate vehicle such as mineral oil, liquid lanolin, or white petrolatum. Sterile ophthalmic gel formulations may be prepared by suspending the compound in a hydrophilic base prepared from the combination of, for example, carbopol-974, or the like, according to the published formulations for analogous ophthalmic preparations; preservatives and tonicity agents can be incorporated.

Compounds in preferred embodiments are contained in a composition in amounts sufficient to prevent, reduce or treat age-related macular degeneration in subjects either susceptible to or having age-related macular degeneration. Such amounts are referred to herein as “an amount effective to prevent, reduce or treat age-related macular degeneration,” or more simply “an effective amount.” The compounds will normally be contained in these formulations in an amount of between about 0.1% and 3.0% (w/v), or between about 0.5 to about 1.5% (w/v). Thus, for topical presentation 1 to 2 drops of these formulations would be delivered to the surface of the eye from 1 to 4 times per day, according to the discretion of a skilled clinician.

The invention will be further illustrated by way of the following Examples.

### EXAMPLE 1

### A Blue Light Damage Model in Balb/C Mice

A blue light damage study in mice was undertaken to evaluate the efficacy of trabodenoson (Compound A), a selective adenosine-A₁ receptor agonist, when given by twice daily topical ocular instillation for 7 days in a mouse model of retinal damage. The rodent retina is widely used to investigate retinal diseases, as well as the response of central nervous system neurons to injury. Light-induced retinal damage (phototoxicity) selectively brings about photoreceptor cell death. Thus, this model is useful for studying the potential mechanisms underlying photoreceptor death and the subsequent retinal degeneration processes since it mimics the photoreceptor degeneration that forms the main characteristic of age-related macular degeneration.

The study design was as follows:

| | | | | Dose | |
|---|---|---|---|---|---|
| Group | | | Dose Level | Concen- | Number |
| No. | Test Material | Route | (μg/eye/day) | tration | of Males |
| 1 | Placebo | Topical | 0 | 0 | 8 |
| 2 | Compound A | Topical | 300 | 1.5% | 8 |
| | Trabodenoson - | | | | |
| | low dose | | | | |
| | (1.5% w/w) | | | | |
| 3 | Compound A | Topical | 600 | 3.0% | 8 |
| | Trabodenoson - | | | | |
| | high dose | | | | |
| | (3.0% w/w) | | | | |
| 4 | BDNF | IVT | 1 | 1 μg/μL | 8 |

BDNF: Brain Derived Neurotrophic Factor;
IVT: intravitreal injection

The Dose Formulation was as follows:

The test item, 1.5% or 3.0% of Compound A (trabodenoson), and placebo were administered to the mice. Aliquots were removed from the refrigerator and allowed to warm to room temperature for at least 30 minutes before dosing. The positive control, BDNF, was reconstituted with 0.9% Sodium Chloride for Injection, USP at a concentration of 1 μg/μL. Any residual volumes were discarded.

Thirty-four (34) male BALB/c mice were received from Charles River, St Constant. The animals were 8 weeks old and weighed between 21.7 and 23.9 g at initiation of dosing.

The animals were group housed at arrival (2 mice/cage), then housed individually following randomization. Animals were housed in wire mesh floor cages or polycarbonate cages containing appropriate bedding. Temperatures of 19° C. to 25° C. with a relative humidity of 30% to 70% were maintained. A 12-hour light/12-hour dark cycle was maintained, except when interrupted for designated procedures. Light intensity was <200 lux in the housing area.

PMI Nutrition International Certified Rodent Chow No. 5CR4 (14% protein) was provided ad libitum throughout the study, except during designated procedures. Municipal tap water after treatment by reverse osmosis and ultraviolet irradiation was freely available to each animal via an automatic watering system (except during designated procedures).

The test item and placebo were administered by twice-daily topical ocular instillation from Days 1 to 7, separated by at least 8 hours. The dose volume for each eye/dose was 10 μL. The formulations were stirred continuously during dose administration and were retained on wet ice between doses.

The positive control, BDNF, was administered to Group 4 animals by single bilateral intravitreal (IVT) injection on Day 1. The dose volume was 1 μL/eye. Animals were anesthetized for the dosing procedure using an isoflurane/oxygen mix and/or a sedative cocktail (ketamine 100 mg/kg; xylazine 10 mg/kg; half or quarter dose if needed given by intraperitoneal injection). Injections were made using a glass microneedle connected to a Hamilton syringe via a sclera pilot hole created with a 30 G needle. A bland lubricating ointment was applied to the eyes following dosing.

On Day 2, animals were transferred to the blue light bins (housed individually). Light fixtures with bulbs emitting blue light (460 to 490 nm; Philips F40/BB Special Blue F40T12/BB) were attached to separate housing racks directly above the cages, such that all animals received similar light exposure. The light intensity in the bins was measured using a light meter; the average intensity was 550 lux. Hiding devices/tubes and bedding were not available in the bins. The exposure period was 7.75 hours for all animals, after which animals were returned to their home cages, at ambient light levels.

Mortality/moribundity checks were performed twice daily, once in the morning and once in the afternoon, throughout the study. Detailed examinations were performed weekly. Individual body weights were measured twice pre-treatment and at necropsy.

Electroretinography (ERG) was performed once pre-treatment and once at the end of Week 1 following overnight dark adaptation. The anesthetic used was ketamine (100 mg/kg) and xylazine (10 mg/kg). Each ERG occasion consisted of a scotopic, single flash stimuli at 0 dB, average of 2 single flashes, a minimum of 120 seconds apart.

All animals were euthanized on Day 8 and underwent exsanguination from the abdominal aorta after carbon dioxide asphyxiation. No necropsy examinations were performed. Both eyes for each animal were retained in Davidson's fixative for at least 24 hours; transferred to 70% Ethanol for at least 18 hours until processing and/or stored in 10% neutral buffered formalin.

One eye from each animal was embedded in paraffin, sectioned, mounted on glass slides, and stained with hematoxylin and eosin. The other eye was retained.

Histopathological evaluation was performed by a board-certified veterinary pathologist or veterinary pathologist with training and experience in laboratory animal pathology. The outer nuclear layer (ONL) thickness (number of cell layers) were counted at 4 locations in the retina and a microscopic evaluation of the retina, including pigmented retinal epithelial cells, was conducted. Representative images were captured for consultation and illustrative purposes.

### Results

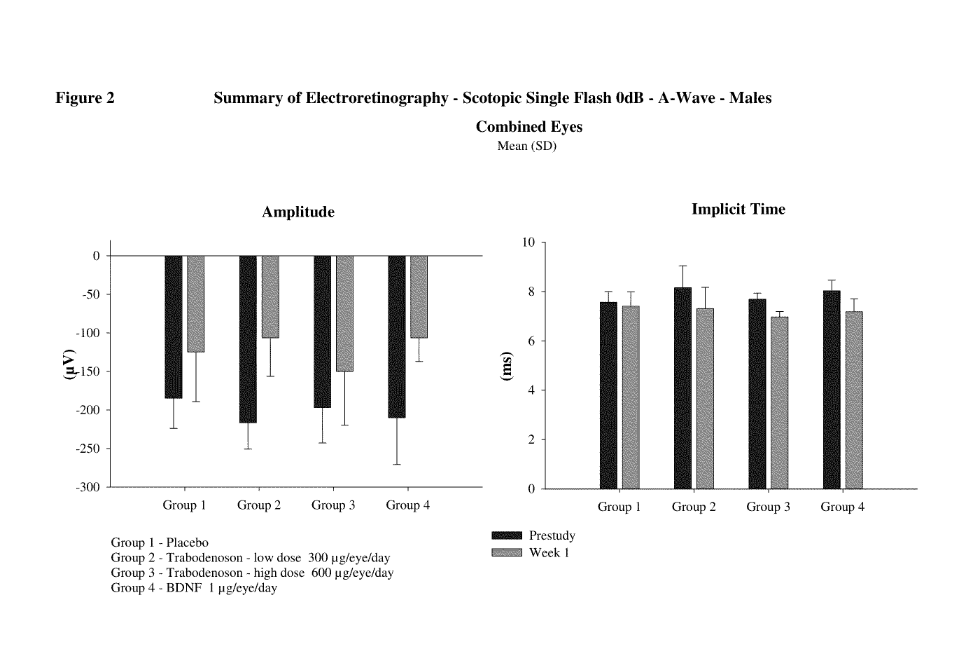

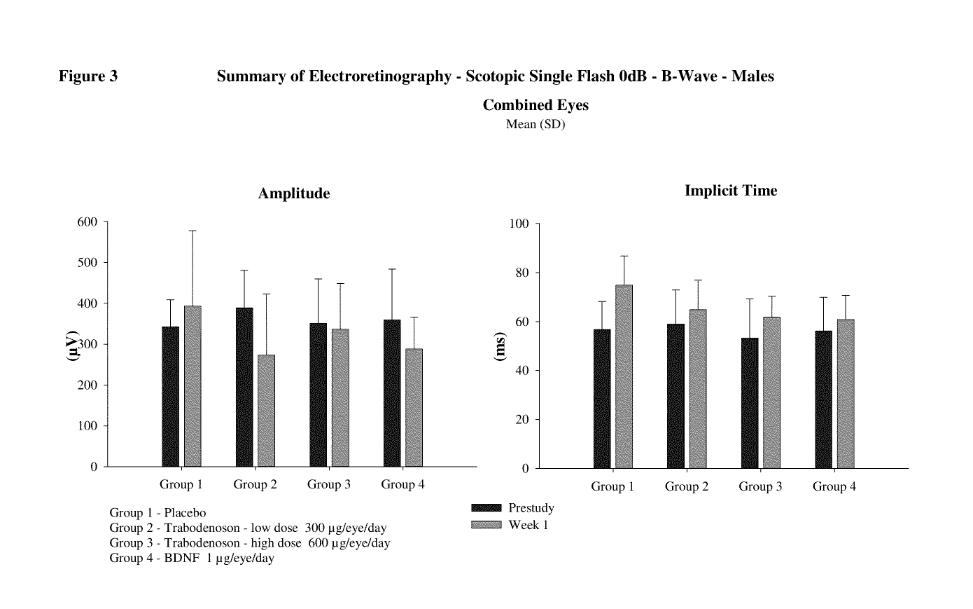

No abnormalities were observed in the general appearance or condition of the animals during the dosing period. One week after blue light exposure, ERG parameters were comparable across treatment groups, including the placebo control group. FIGS. 2 and 3. At 300 μg/eye/day, a minimal to moderate decrease in cellularity of the retina (7/8 animals) was noted. The incidence and severity of this finding was similar to that observed in the placebo control (6/8 animals). The decreased cellularity in the retina notably affected the central region of the outer nuclear cell layer, with reduced thickness of the outer plexiform and photoreceptor layers. In a few of the most affected animals, this change was associated with deposits of basophilic pigment in the outer nuclear layer, likely related to the cellular degeneration and loss. At 600 μg/eye/day, retinal cellularity was either minimally decreased (2/8) or not affected at all, similar to BDNF-treated animals.

Group mean of the number of cell layers in retinal outer nuclear layer were slightly higher in the 600 μg/eye/day and BDNF groups (8.56 and 8.75 layers, respectively) compared to the placebo and 300 μg/eye/day groups (7.22 and 7.28 layers, respectively); however, a large range was observed in individual animals within the groups as shown in the following table.

Individual Eye Retinal Outer Nuclear Layer Cellularity
Males

| | Animal | Retinal outer nuclear layer thickness |
|---|---|---|
| Group | Number | (Mean number of nuclei)* |
| 1 | 1001 | 11.25 |
| | 1002 | 7.75 |
| | 1003 | 5.75 |
| | 1004 | 8.50 |
| | 1005 | 5.25 |
| | 1006# | 7.00 |
| | 1007 | 6.00 |
| | 1008# | 6.25 |
| 2 | 2001 | 10.00 |
| | 2002 | 9.25 |
| | 2003 | 6.50 |
| | 2004 | 5.25 |
| | 2005 | 7.25 |
| | 2006 | 6.25 |
| | 2007 | 5.50 |
| | 2008 | 8.25 |
| 3 | 3001 | 9.25 |
| | 3002 | 9.00 |
| | 3003 | 9.75 |
| | 3004 | 7.75 |
| | 3005 | 9.50 |
| | 3006 | 8.75 |
| | 3007# | 6.75 |
| | 3008# | 7.75 |
| 4 | 4001 | 7.75 |
| | 4002 | 9.25 |
| | 4003 | 8.50 |
| | 4004 | 8.75 |
| | 4005# | 7.75 |
| | 4006 | 10.50 |
| | 4007 | 8.50 |
| | 4008 | 9.00 |

*Mean of 4 measurements per retina
#Measurements were made on the left eye
Group 1 - Placebo
Group 2 - Compound A (Trabodenoson) - low dose 300 μg/eye/day
Group 3 - Compound A (Trabodenoson) - high dose 600 μg/eye/day
Group 4 - BDNF 1 μg/eye/day

Based on the absence of any ERG changes and the limited microscopic changes in the retina of placebo animals, the blue light exposure in this study (550 lux for 7.75 hours) did not efficiently induce cell loss in the outer nuclear layer retina. However, a reduced incidence and severity of decreased retinal cellularity are indicative of a protective effect following 7 days of twice daily Compound A (trabodenoson) administration at 600 μg/eye/day by topical ocular instillation.

### EXAMPLE 2

### A Blue Light Damage Model in Balb/C Mice

A second blue light damage study in mice was undertaken to further evaluate the efficacy of trabodenoson (Compound A), a selective adenosine-A₁ receptor agonist, when given by twice or three time daily topical ocular instillation for 7 days in a mouse model of retinal damage. The rodent retina is widely used to investigate retinal diseases, as well as the response of central nervous system neurons to injury. Light-induced retinal damage (phototoxicity) selectively brings about photoreceptor cell death. Thus, this model is useful for studying the potential mechanisms underlying photoreceptor death and the subsequent retinal degeneration processes since it mimics the photoreceptor degeneration that forms the main characteristic of age-related macular degeneration.

The study design was as follows:

| | | | | Dose | |
|---|---|---|---|---|---|
| Group | | | Dose Level | Concen- | Number |
| No. | Test Material | Route | (μg/eye/day) | tration | of Males |
| 1 | Placebo | Topical | 0 | 0 | 8 |
| 2 | Compound A | Topical | 600 | 3.0% | 8 |
| | Trabodenoson - | | | | |
| | low dose | | | | |
| | (3.0% w/w) | | | | |
| 3 | Compound A | Topical | 900 | 3.0% | 8 |
| | Trabodenoson - | | | | |
| | high dose | | | | |
| | (3.0% w/w) | | | | |
| 4 | BDNF | IVT | 1 | 1 μg/μL | 8 |

BDNF: Brain Derived Neurotrophic Factor;
IVT: intravitreal injection

The Dose Formulation was as follows:

The test item, 3.0% of Compound A (trabodenoson), and placebo were administered to the mice. Aliquots were removed from the refrigerator and allowed to warm to room temperature for at least 30 minutes before dosing. The positive control, BDNF, was reconstituted with 0.9% Sodium Chloride for Injection, USP at a concentration of 1 μg/μL. Any residual volumes were discarded.

Thirty-four (34) male BALB/c mice were received from Charles River, St Constant. The animals were 9 weeks old and weighed between 21.8 and 26.4 g at initiation of dosing.

The animals were group housed at arrival (2 mice/cage), then housed individually following randomization. Animals were housed in wire mesh floor cages or polycarbonate cages containing appropriate bedding. Temperatures of 19° C. to 25° C. with a relative humidity of 30% to 70% were maintained. A 12-hour light/12-hour dark cycle was maintained, except when interrupted for designated procedures. Light intensity was <200 lux in the housing area.

PMI Nutrition International Certified Rodent Chow No. 5CR4 (14% protein) was provided ad libitum throughout the study, except during designated procedures. Municipal tap water after treatment by reverse osmosis and ultraviolet irradiation was freely available to each animal via an automatic watering system (except during designated procedures).

| | | | | Dose | |
|---|---|---|---|---|---|
| Group | | | Dose Level | Concen- | Number |
| No. | Test Material | Route | (μg/eye/day) | tration | of Males |
| 1 | Placebo | Topical | 0 | 0 | 8 |
| 2 | Trabodenoson - | Topical | 600 | 3.0% | 8 |
| | low dose | | | | |
| 3 | Trabodenoson - | Topical | 900 | 3.0% | 8 |
| | high dose | | | | |
| 4 | BDNF | IVT | 1 | 1 μg/μL | 8 |

The test item and placebo were administered by twice-daily topical ocular instillation from Days 1 to 8, separated by at least 8 hours. Groups 1 and 2 were dosed twice daily and Group 3 was dose three times daily, with a minimum of 8 hours between the first and last dose. The dosing period was extended by one day to 8 days to allow for electroretinography (ERG), as overnight dark adaptation prior to Day 7 was interrupted by lights automatically turning on in the animal room. The dose volume for each eye/dose was 10 μL. The formulations were stirred continuously during dose administration and were retained on wet ice between doses.

The positive control, BDNF, was administered to Group 4 animals by single bilateral intravitreal (IVT) injection on Day 1. The dose volume was 1 μL/eye. Animals were anesthetized for the dosing procedure using an isoflurane/oxygen mix and/or a sedative cocktail (ketamine 100 mg/kg; xylazine 10 mg/kg; half or quarter dose if needed given by intraperitoneal injection). Injections were made using a glass microneedle connected to a Hamilton syringe via a sclera pilot hole created with a 30 G needle. A bland lubricating ointment was applied to the eyes following dosing.

On Day 2, animals were transferred to the blue light bins (housed individually). Light fixtures with bulbs emitting blue light (460 to 490 nm; Philips F40/BB Special Blue F40T12/BB) were attached to separate housing racks directly above the cages, such that all animals received similar light exposure. The light intensity in the bins was measured using a light meter; the average intensity was 1100 lux. Hiding devices/tubes and bedding were not available in the bins. The exposure period was 6 hours for all animals, after which animals were returned to their home cages, at ambient light levels.

Mortality/moribundity checks were performed twice daily, once in the morning and once in the afternoon, throughout the study. Detailed examinations were performed weekly. Individual body weights were measured once pre-treatment.

Electroretinography (ERG) was performed once pre-treatment and once at the end of the 8-day treatment period following overnight dark adaptation. The anesthetic used was ketamine (100 mg/kg) and xylazine (10 mg/kg). Each ERG occasion consisted of a scotopic, single flash stimuli at 0 dB, average of 2 single flashes, a minimum of 120 seconds apart. The results of the ERG

All animals were euthanized on Day 8 and underwent exsanguination from the abdominal aorta after carbon dioxide asphyxiation. Both eyes for each animal were collected and retained in Davidson's fixative for at least 24 hours; transferred to 70% Ethanol for at least 18 hours until processing and/or stored in 10% neutral buffered formalin.

One eye from each animal was embedded in paraffin, sectioned, mounted on glass slides, and stained with hematoxylin and eosin.

Histopathological evaluation was performed by a board-certified veterinary pathologist or veterinary pathologist. The outer nuclear layer (ONL) thickness (number of cell layers) were counted at 4 locations in the retina and a microscopic evaluation of the retina, including pigmented retinal epithelial cells, was conducted.

### Results

One week after blue light exposure, ERG results were suggestive of a protective effect against blue light damage.

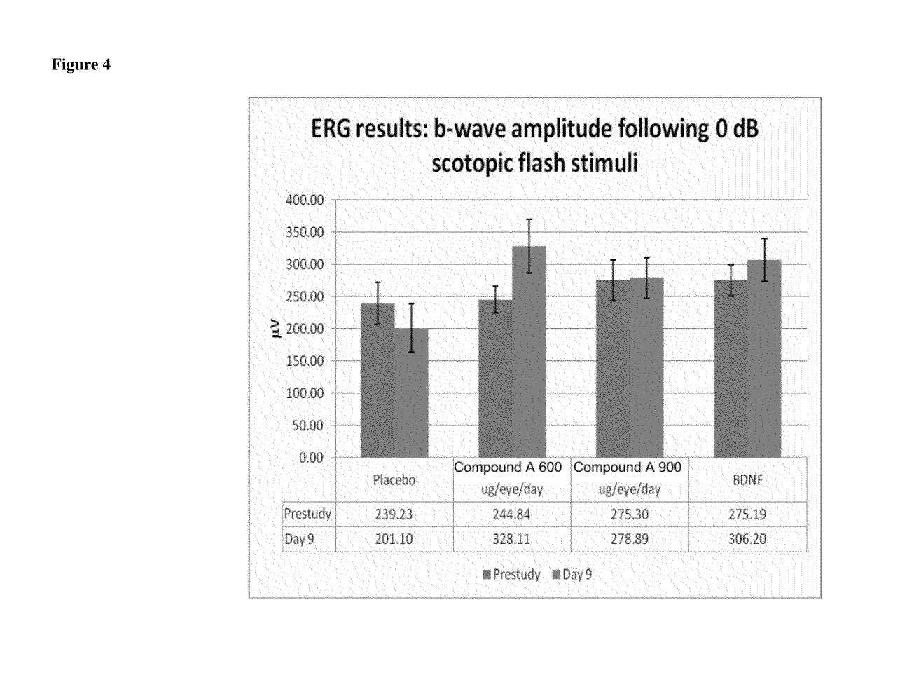

B-wave amplitude following 0 dB scotopic flash stimuli was higher in Compound A groups (600 or 900 μg/eye/day) than the placebo control and comparable to the BDNF positive control—see FIG. 4.

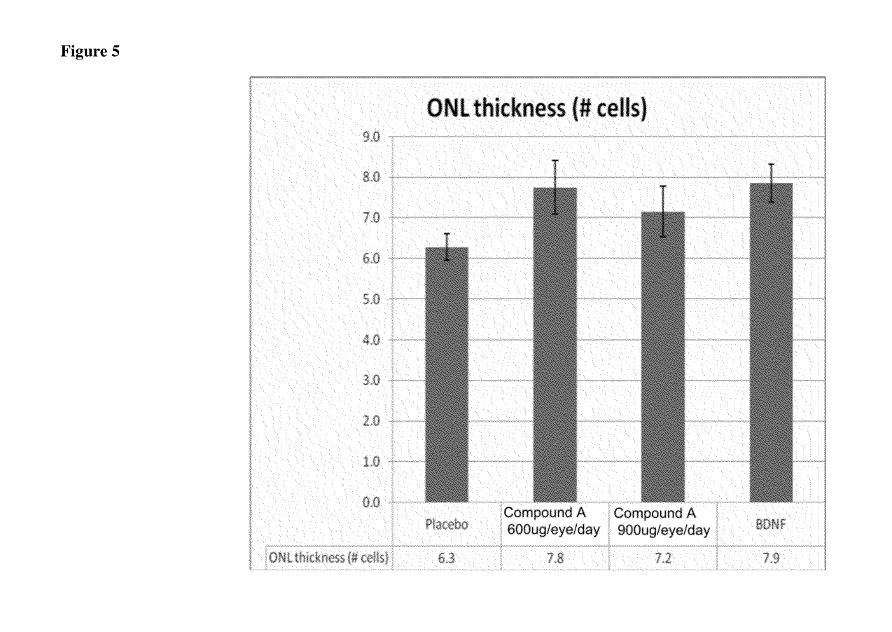

When examined microscopically, the average number of cell layers in the ONL was reduced in the placebo group, when compared to the 600 or 900 μg Compound A/eye/day groups and the BDNF control—see FIG. 5. Overall, the Compound A-low dose group retained the greatest number of nuclear bodies in the ONL based on the average ONL thickness counts. The changes in the number of ONL cells correlated with the ERG results. Other microscopic observations in the cornea and/or sclera were observed more frequently in the placebo control group.

Overall, administration of Compound A two or three-times daily for 7 days at 600 or 900 μg/eye/day by topical ocular instillation resulted in a preservation of ERG function and cellularity in the ONL layer of the retina following the blue light exposure (1100 lux for 6 hours) on Day 2. These results are indicative of a protective effect of Compound A in this mouse model of retinal damage.

### EXAMPLE 3

### Compound Synthesis

2′, 3′-Isopropylidene-N⁶-cyclohexyladenosine: A solution of 6-chloroadenosine (2.58 g) and cyclohexylamine (5 g) in ethanol (20 ml) was heated at reflux for 6 hours then cooled to room temperature. The reaction mixture was concentrated in vacuo and the resultant residue was diluted with water (50 ml) and ethyl acetate (300 ml). The organic layer was separated and the aqueous layer was extracted with ethyl acetate (2×50 ml). The combined organic layers were washed with water (1×30 ml), dried over sodium sulfate, concentrated in vacuo and dried under vacuum to provide N⁶-cyclohexyladenosine as a white solid (2.600 g). N⁶-Cyclohexyladenosine (2.6 g) was diluted with acetone (30 ml) and to the resultant solution was added 2, 2-dimethoxypropane (12 ml), followed by D-camphorsulphonic acid (3.01 g) and the mixture was allowed to stir at room temperature for 18 hours. The reaction mixture was concentrated in vacuo and the resultant residue was diluted with ethyl acetate (150 ml), then neutralized to pH 8.0 using saturated aqueous NaHCO₃. The organic layer was separated, dried over sodium sulfate, concentrated in vacuo. The residue was purified twice on the silica gel column using MeOH— CH₂Cl₂ (4:96) as an eluent to provide 2′, 3′-isopropylidene-N⁶-cyclohexyladenosine (3.16 g). ¹H NMR (CDCl₃g): δ 6 1.23-1.47 (m, 9H), 1.38 (s, 3H), 1.64 (s, 3H), 1.79-1.81 (m, 1H), 2.04-2.06 (m, 1H), 3.80 (d, J=12 Hz, 1H), 3.96 (d, J=12 Hz, 1H), 4.53 (s, 1H), 5.09-5.16 (m, 2H), 5.80-5.92 (m, 2H), 7.79 (s, 1H), 8.24 (s, 1H), 8.22-8.38 (m, 1H).

N⁶-Cyclohexyladenosine-5′-O-nitrate (Compound E): Acetic anhydride (6 ml) was slowly added to a stirred solution of nitric acid (2 g, 63%) at −25° C. (CCl₄-CO₂ bath used for cooling) and the reaction temperature maintained at −7.5 to 0° C. for additional 1 hr. A solution of 2′, 3′-isopropylidene-N⁶-cyclohexyladenosine (1.0 g) in acetic anhydride (3 mL) was added slowly. The resultant reaction was allowed to stir at 0 to −5° C. for 2 hour and the mixture was slowly poured slowly into an ice-cold solution of aqueous NaHCO₃ (40 mL) and ethyl acetate (150 mL) and it was allowed to stir for 5 minutes. The organic layer was separated and washed with water, dried over sodium sulfate, and concentrated in vacuo. The residue was diluted with a mixture of TFA (16 mL) and water (4 mL) and the mixture was allowed to stir for 30 minutes at room temperature. The mixture was concentrated in vacuo and the resultant residue was diluted with water (10 mL) and concentrated in vacuo. The residue obtained was diluted with ethyl acetate and washed with saturated aqueous sodium bicarbonate, and the organic layer was dried over sodium sulfate and concentrated in vacuo. The residue was purified on the silica gel column using ethyl acetate hexane (from 40:60 to 20:80 gradient) to provide N⁶-cyclohexyladenosine-5′-O-nitrate (0.150 gm). ¹H NMR (DMSO-D₆): δ 6 1.08-1.13 (m, 1H), 1.27-1.41 (m, 4H), 1.57-1.83 (m. 6H), 4.12-4.17 (m, 2H), 4.30-4.33 (m, 1H), 5.48 (d, J=5.4 Hz, 1H), 5.60 (d, J=5.7 Hz, 1H), 5.90 (d, J=4.8 Hz, 1H), 7.59 (d, J=8.1 Hz, 1H), 8.16 (s, 1H), 8.29 (s, 1H).

N⁶-(exo-2-Norbornyl)adenosine-5′-O-nitrate (Compound F): 2′, 3′-Isopropylidene-N⁶-exo-norbornyladenosine was prepared following the procedure of 2′, 3′-isopropylidene-N⁶-cyclohexyladenosine and used for the subsequent reaction. Acetic anhydride (6 ml) was slowly added to a stirred solution of nitric acid (2 g, 63%) at −25° C. (CCl₄-CO₂ bath used for cooling) and the reaction temperature maintained at −7.5 to 0° C. for additional 1 hr. A solution of 2′, 3′-isopropylidene-N⁶-exo-norbornyladenosine (1.2 g) in acetic anhydride (3 mL) was added slowly. The mixture was allowed to stir at 0 to −5° C. for 40 minutes and the mixture was slowly poured slowly into an ice-cold solution of aqueous NaHCO₃ (40 mL). The solution was extracted in dichloromethane. The organic layer was separated and washed with brine, dried over sodium sulfate, and concentrated under vacuo. The residue was purified on the silica gel column using ethyl acetate-hexane (1:1) to provide the desired product (0.245 g) and the starting compound (1.0 g). The nitro product (0.245 g) was diluted in a mixture of TFA (15 mL) and water (5 mL) and the mixture was allowed to stir for 30 minutes at room temperature. It was concentrated under vacuo and diluted with water (10 mL) and concentrated in vacuo. The resultant residue was diluted with ethyl acetate and washed with saturated aqueous sodium bicarbonate. The organic layer was dried over sodium sulfate and concentrated in vacuo. The residue was recrystallized from the mixture of ethyl acetate and hexane to provide N⁶-exo-2-norbornyladenosine-5′-O-nitrate (0.123 gm). ¹H NMR (DMSO-D₆): δ 1.03-1.21 (m, 3H), 1.40-1.56 (m, 3H), 1.58-1.64 (m. 4H), 3.94 (bs, 1H), 4.13-4.17 (m, 1H), 4.30 (bs, 1H), 4.66-4.87 (m, 3H), 5.49 (d, J=5.4 Hz, 1H), 5.62 (d, J=5.4 Hz, 1H), 5.91 (d, J=4.8 Hz, 1H), 7.60 (d, J=6.6 Hz, 1H), 8.20 (s, 1H), 8.31 (s, 1H).

2-Chloro-N⁶-cyclohexyladenosine: A mixture of 2,6-dichloroadenosine (1.0 g) and cyclohexylamine (0.926 g) in ethanol (30 ml) was heated at reflux for 6 hours then cooled to room temperature. The mixture was concentrated under vacuo. The residue was purified on the silica gel column using MeOH—CH₂Cl₂ (1:6 to 1:5). The combined fractions were concentrated and dried under vacuum to provide 2-chloro-N⁶-cyclohexyladenosine as a white solid (2.600 g). ¹H NMR (DMSO-D₆): δ 1.12-1.21 (m, 2H), 1.33-1.43 (m, 3H), 1.63-1.86 (m, 6H), 3.57-3.62 (m, 1H), 3.66-3.69 (m, 1H), 3.97 (d, J=3 Hz, 1H), 4.16 (d, J=3.3 Hz, 1H), 4.54 (d, J=5.4 Hz, 1H), 5.08-5.11 (m, 1H), 5.24 (d, J=4.8 Hz, 1H), 5.51 (d, J=5.7 Hz, 1H), 5.85 (d, J=5.7 Hz, 1H), 8.26 (d, J=8.4 Hz, 1H), 8.41 (s, 1H).

2-Chloro-2′, 3′-isopropylidene-N⁶-cyclohexyladenosine: 2-Chloro-N⁶-cyclohexyladenosine (0.5 g) was diluted with acetone (30 ml) and to the mixture was added 2,2-dimethoxypropane (2.04 g), followed by D-camphorsulphonic acid (CSA, 0.272 g). The resultant reaction mixture was allowed to stir at room temperature for 2 hours. Additional CSA (0.2 g) was added and stirred for 2 hours. The mixture was concentrated in vacuo and the resultant residue was diluted with ethyl acetate, then neutralized to pH 8.0 using concentrated aqueous NaHCO₃. The organic layer was separated, dried over sodium sulfate, concentrated under vacuum to provide 2-chloro-2′, 3′-isopropylidene-N⁶-cyclohexyladenosine (0.378 g). ¹H NMR (CDCl₃): δ 6 1.23-1.30 (m, 3H), 1.36-1.44 (m, 1H), 1.63 (s, 3H), 1.68-1.79 (m, 5H), 2.04-2.08 (m, 2H), 3.81 (d, J=5 Hz, 1H), 3.99 (d, J=12.9 Hz, 1H), 4.51 (s, 1H), 5.11 (d, J=5.7 Hz, 1H), 5.15-5.18 (m, 1H), 5.75 (bs, 1H), 5.78 (d, J=4.5 Hz, 1H), 5.96 (bs, 1H), 7.76 (s, 1H).

2-Chloro-N⁶-cyclohexyladenosine-5′-O-sulfate sodium salt (Compound G): 2-Chloro-2′, 3′-isopropylidene-N⁶-cyclohexyladenosine (0.540 g) was dissolved in DMF (6 ml) and added slowly in to the solution of sulfur trioxide (0.302 g) in DMF (3 ml). The mixture was stirred overnight at room temperature. It was concentrated on rotavaporator and the residue was diluted with water (8 ml). The water solution was slowly neutralized with NaOH (0.1 N) to pH 7.0. It was extracted in ethyl acetate and the aqueous layer was then concentrated. The white solid obtained was used as such for the next step. The protected sodium sulfate salt was treated with the mixture of TFA-water (16:4 ml) and stirred for 30 min. The reaction mixture was concentrated and the residue was crystallized from acetone to provide 2-chloro-N⁶-cyclohexyladenosine-5′-O-sulfate sodium salt (0.150 g). ¹H NMR (DMSO-D₆): δ 1.10-1.13 (m, 1H), 1.25-1.41 (m, 4H), 1.57-1.83 (m. 6H), 3.72-4.08 (m, 4H), 4.47 (s, 1H), 5.81 (s, 1H), 8.14 (d, J=6.0 Hz, 1H), 8.43 (s, 1H).

2-Chloro-N⁶-cyclohexyladenosine-5′-O-nitrate (Compound H): Following the nitration and the TFA water deprotection reactions, 2-chloro-N⁶-cyclohexyladenosine-5′-O-nitrate was prepared from 2-chloro-2′, 3′-isopropylidene-N⁶-cyclohexyladenosine. ¹H NMR (CDCl₃): δ 1.06-1.42 (m, 4H), 1.64-1.88 (m, 5H), 4.08 (bs, 1H), 4.21 (s, 1H), 4.30 (d, J=4.2 Hz, 1H), 4.41 (s, 1H), 4.83-4.88 (m, 2H), 5.57 (d, J=5.4 Hz, 1H), 5.70 (d, J=4.5 Hz, 1H), 5.90 (d, J=5.1 Hz, 1H), 8.26 (d, J=8.7 Hz, 1H), 8.38 (s, 1H).

### Synthesis of Compound A

N⁶-Cyclopentyladenosine (Compound I): A solution of 6-chloroadenosine (43 g) and cyclopentylamine (5 eq.) in ethanol (50 eq.) was heated at reflux for 3 hours then cooled to room temperature. The resultant reaction mixture was concentrated in vacuo and the resultant residue was diluted with water (400 ml) and ethyl acetate (400 ml). The organic layer was separated and the aqueous layer was extracted into ethyl acetate (2×400 ml). The combined organic layers were washed with water (2×200 ml), dried over sodium sulfate, concentrated in vacuo and dried under vacuum to provide a solid which was suspended in MeOH (400 mL), filtered and dried to provide N⁶-cyclopentyladenosine (43.8 g).

2′, 3′-isopropylidene-N⁶-cyclopentyladenosine: N⁶-cyclopentyladenosine (43 g) was diluted with acetone (75 eq.) and to the resultant solution was added 2,2-dimethoxypropane (5 eq.), followed by D-camphorsulphonic acid (1 eq) and the resultant reaction was allowed to stir at room temperature for 3 hours. The resultant reaction mixture was concentrated in vacuo and the resultant residue was diluted with ethyl acetate, then neutralized to pH 7.0 using concentrated aqueous NaHCO₃. The organic layer was separated, dried over sodium sulfate, concentrated in vacuo and dried under vacuum to provide a solid which was suspended in hexane (250 mL), filtered, washed with hexane and dried under vacuum to provide 2′, 3′-isopropylidene-N⁶-cyclopentyl adenosine (43 g).

2′, 3′-isopropylidene-N⁶-cyclopentyladenosine-5′-nitrate: Acetic anhydride (22 eq) was slowly added to a stirred solution of nitric acid (5 eq., 63%) at −10° C. (acetonitrile-CO₂ bath used for cooling) over a period of 4 hours with the reaction temperature maintained at −5 to 5° C. during the addition. The resultant solution was cooled to −20° C. and a solution of 2′, 3′-isopropylidene-N⁶-cyclopentyladenosine (18.250 gm, 0.048 mol) in acetic anhydride (37 mL, 8 eq.) was added slowly. The resultant reaction was allowed to stir at −15 to −5° C. for 1 hour and the resultant reaction mixture was slowly poured slowly into an ice-cold solution of aqueous NaHCO₃ (168 gm in 800 mL water) and ethyl acetate (350 mL) and the resultant solution was allowed to stir for 5 minutes. The organic layer was separated and the aqueous layer was extracted using ethyl acetate (350 mL). The combined organic layers were washed with water, and dried over sodium sulfate, concentrated in vacuo and purified using flash column chromatography on silica gel using 70% ethyl acetate-hexane as eluent to provide 2′, 3′-isopropylidene-N⁶-cyclopentyladenosine-5′-nitrate (14.9 g).

Compound A: 2′, 3′-isopropylidene-N⁶-cyclopentyladenosine-5′-nitrate (4.8 g) was diluted with a mixture of TFA (20 mL) and water (5 mL) and the resultant reaction was allowed to stir for 30 minutes at room temperature. The resultant reaction mixture was concentrated in vacuo and the resultant residue was diluted with water (10 mL) and concentrated in vacuo. The resultant residue was diluted with ethyl acetate and washed with saturated aqueous sodium bicarbonate, and the organic layer was dried over sodium sulfate and concentrated in vacuo to provide a white solid residue which was dried under vacuum and then recrystallized from cold ethanol to provide Compound A (3.1 gm). ¹H-NMR (DMSO-d₆): δ 1.49-1.58 (m, 4H), 1.66-1.72 (m, 2H), 1.89-1.94 (m, 2H), 4.12-4.17 (m, 1H), 4.28-4.33 (m, 1H), 4.48 (bs, 1H), 4.65-4.87 (m, 3H), 5.5 (d, J=5.1 Hz, 1H), 5.63 (d, J=5.7 Hz, 1H), 5.91 (d, J=5.1 Hz, 1H), 7,75 (d, J=7.5 Hz, 1H), 8.17 (bs, 1H), 8.30 (s, 1H); MS (ES⁺): m/z 381.35 (M+1); Anal. Calcd for C₁₅H₂₀N₆O₆: C, 47.37; H, 5.30; N, 22.10; Found: C, 47.49; H, 5.12, N, 21.96.

### Synthesis of Compound B

2-Chloro-N⁶-cyclopentyladenosine-2′, 3′, 5′-triacetoxy-2,6-dichloroadenosine (1.5 g) and cyclopentylamine (8 eq.) were diluted with ethanol (50 eq.) and the resulting solution was heated at reflux for about 15 hours, then cooled to room temperature and concentrated in vacuo to provide a crude residue which was diluted with a mixture of ethyl acetate and water and transferred to a separatory funnel. The organic layer was separated, dried over sodium sulfate and concentrated in vacuo to provide a crude residue which was purified using flash column chromatography on silica gel (8% MeOH-dichloromethane as eluent) to provide 2-chloro-N⁶-cyclopentyladenosine (0.948 g). MS m/z 370.32 [M+H]⁺.

2′, 3′-Isopropylidene-2-chloro-N⁶-cyclopentyladenosine: 2-chloro-N⁶-cyclopentyladenosine (900 mg, as prepared in the previous step) and 2,2-dimethoxypropane (10 eq.) were diluted with acetone (15 mL) and to the resulting solution was added D-camphorsulphonic acid (1 eq) and the resulting reaction was allowed to stir at room temperature for 2 hr. The resulting reaction mixture was concentrated in vacuo, diluted with a mixture of saturated aqueous NaHCO₃ and ethyl acetate, and transferred to a separatory funnel. The organic layer was separated, dried over sodium sulfate and concentrated in vacuo to provide a crude residue which was purified using flash column chromatography on silica gel (using 5% MeOH-dichloromethane as eluent) to provide 2′, 3′-Isopropylidene-2-chloro-N⁶-cyclopentyladenosine (0.905 g). ¹H NMR (CDCl₃, 300 MHz): δ 1.36 (s, 3H), 1.62 (s, 3H), 1.66-2.16 (m, 9H), 3.78 (d, J=12.9 Hz, 1H), 3.98 (d, J=12.9 Hz, 1H), 4.51 (bs, 1H), 4.55-4.60 (m, 1H), 5.09-5.17 (m, 2H), 5.81 (bs, 1H), 7.25 (s, 1H), 7.89 (s, 1H).

2′, 3′-Isopropylidene-2-chloro-N⁶-cyclopentyladenosine-5′-nitrate: A solution of nitric acid (2.0 mL, 60%) was added slowly over a period of 30 minutes to acetic anhydride (16.0 mL) at −10 to 10 ° C. (using acetonitrile-CO₂ cooling bath) and the reaction mixture was allowed to stir at −10 to 10 ° C. for 10 minutes. The reaction mixture was then cooled to −30 ° C. and then a solution of 2′, 3′-Isopropylidene-2-chloro-N⁶-cyclopentyladenosine (655 mg, 0.0016 mol, as prepared in the previous step) in acetic anhydride (8.0 mL) was added slowly. When addition was complete, the resulting reaction was allowed to warm to −5 ° C. and monitored using TLC (solvent 5% MeOH—CH₂Cl₂ or 70% EtOAc-hexane). When the reaction was complete, the reaction mixture was poured slowly into an ice cold mixture of saturated aqueous NaHCO₃ (300 equivalent in 75 mL water) and ethyl acetate (60 mL). The organic layer was separated and the aqueous layer was back extracted with ethyl acetate. The combined organic layers were washed with water, dried over sodium sulfate, and concentrated in vacuo to provide a crude residue. The crude residue was purified using flash column (5% methanol-dichloromethane as eluent) to provide 2′, 3′-Isopropylidene-2-chloro-N⁶-cyclopentyladenosine-5′-nitrate (0.435 g). ¹H NMR (CDCl₃, 300 MHz): δ 1.38 (s, 3H), 1.59 (s, 3H), 1.66-2.13 (m, 9H), 4.50-4.55 (m, 1H), 4.71-4.83 (m, 2H), 5.14-5.17 (m, 1H), 5.31 (d, J=5.7 Hz, 1H), 6.04 (s, 1H), 7.24 (s, 1H), 7.81 (s, 1H). MS m/z 455.44 [M+H]⁺.

Compound B: 2′, 3′-Isopropylidene-2-chloro-N⁶-cyclopentyladenosine-5′-nitrate (0.435 g, as prepared in the previous step) was diluted with TFA (20 mL) and water (5 mL) and the resulting solution was allowed to stir for 30 minutes. The resulting reaction mixture was concentrated in vacuo and the resulting residue was diluted with water (10 mL) and the resulting solution was concentrated in vacuo. The crude residue obtained was diluted with ethyl acetate, transferred to a separatory funnel, washed with saturated aqueous sodium bicarbonate, dried over sodium sulfate and concentrated in vacuo. The crude residue obtained was purified using flash column chromatography on silica gel (using 10% methanol-dichloromethane as eluent) to provide Compound 16 (0.250 g). ¹H NMR (DMSO-d₆, 300 MHz): δ 1.52-1.95 (m, 9H), 4.13-4.24 (m, 2H), 4.55-4.58 (m, 1H), 4.73-4.85 (m, 2H), 5.50 (bs, 1H), 5.61 (bs, 1H), 5.84 (d, J=5.1 Hz, 1H), 8.33 (bs, 2H), MS m/z 414.85[M+H]⁺.

### Synthesis of Compound C (Sodium Salt)

A mixture of 2′, 3′-isopropylidene-N⁶-cyclopentyladenosine (1 g, 0.0026 mol, prepared as set forth in Example 1) and sulfur trioxide-pyridine complex (0.0039 mol) in DMF (17 mL) was stirred at room temperature for about 18 hours. The DMF was removed in vacuo and the resulting residue was dried in vacuo. The dried residue was diluted with water (25 mL), neutralized to pH 7.0 using NaOH (1N) and concentrated in vacuo to provide a crude residue which was diluted with an solution of TFA (80% solution in water, 50 mL). The resulting solution was allowed to stir at 25 ° C. for 30 minutes and the reaction mixture was concentrated in vacuo to afford a crude residue which was diluted with water (10 mL) and concentrated in vacuo. The crude compound obtained was recrystallized from acetone-water to provide compound C (sodium salt) (805 mg). ¹HMNR (DMSO-d₆, 300 MHz): 1.53-1.96 (m, 9H), 3.78-4.10 (m, 4H), 4.43-4.54 (m, 2H), 5.90 (d, J=5.1 Hz, 1H), 8.23 (s, 1H), 8.46 (s, 1H). MS m/z 416.20 [M+H]⁺.

### EXAMPLE 4

### Binding Studies

### Cell Culture and Membrane Preparation

CHO cells stably transfected with human adenosine Aₗ receptor are grown and maintained in Dulbecco's Modified Eagles Medium with nutrient mixture F12 (DMEM/F12) without nucleosides, containing 10% fetal calf serum, penicillin (100 U/mL), streptomycin (100 μg/mL), L-glutamine (2 mM) and Geneticin (G-418, 0.2 mg/mL; A_{2B}, 0.5 mg/mL) at 37° C. in 5% CO₂/95% air. Cells are then split 2 or 3 times weekly at a ratio of between 1:5 and 1:20.

Membranes for radioligand binding experiments are prepared from fresh or frozen cells as described in Klotz et al., Naunyn-Schmiedeberg's Arch. Pharmacol., 357:1-9 (1998). The cell suspension is then homogenized in ice-cold hypotonic buffer (5 mM Tris/HCl mM EDTA, pH 7.4) and the homogenate is spun for 10 minutes (4° C.) at 1,000 g. The membranes are then sedimented from the supernatant for 30 minutes at 100,000 g and resuspended in 50 mM Tris/HCl buffer pH 7.4 (for A₃ adenosine receptors: 50 mM Tris/HCl 10 mM MgCl₂, 1 mM EDTA, pH 8.25), frozen in liquid nitrogen at a protein concentration of 1-3 mg/mL and stored at −80° C.

### Adenosine Receptor Binding Studies

The affinities of selected Purine Compounds for the adenosine A₁ receptor can be determined by measuring the displacement of specific [³H] 2-chloro-N⁶-cyclopentyl adenosine binding in CHO cells stably transfected with human recombinant A₁ adenosine receptor expressed as Ki (nM).

Dissociation constants of unlabeled compounds (Kᵢ-values)are determined in competition experiments in 96-well microplates using the A₁ selective agonist 2-chloro-N⁶-[³H]cyclopentyladenosine ([³H]CCPA, 1nM) for the characterization of A₁ receptor binding. Nonspecific binding is determined in the presence of 100 μM R-PIA and 1 mM theophylline, respectively. For details see Klotz et al., Naunyn-Schmiedeberg's Arch. Pharmacol., 357:1-9, 1998. Binding data can be calculated by non-linear curve fitting using the program SCTFIT (De Lean et al. Mol. Pharm. 1982, 21:5-16).

### Functional Characterization

The A₁ and A₃ receptor-mediated inhibition of forskolin-stimulated adenylyl cyclase activity was tested in membranes prepared from CHO cells stably transfected with the human A₁ and A₃ adenosine receptors. The A_{2A} and A_{2B} receptor-mediated stimulation of basal cyclase activity was tested in membranes prepared from CHO cells stably transfected with the human A_{2A} and A₃ adenosine receptors.

Adenylyl cyclase inhibition via human
adenosine A₁ and A₃ receptors

| | A₁ | A₃ |
|---|---|---|
| | (EC₅₀ nM) | (EC₅₀ nM) |
| Compound A | 17 | >100,000 |
| Compound B | 20 | >100,000 |
| Compound E | 29 | >100,000 |
| Compound G | 19 | >100,000 |

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

1. A method of preventing age-related macular degeneration in a subject in need thereof, comprising the step of: applying an effective amount of an ophthalmic pharmaceutical composition comprising a compound according to Formula I to an eye of the subject, 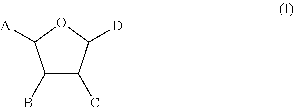
or a pharmaceutically acceptable salt thereof,
wherein
A is —CH₂ONO₂, —CH₂OH, —CH₂OSO₃H;
B and C are —OH;
D is 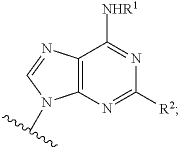
A and B are trans with respect to each other;
B and C are cis with respect to each other;
C and D are cis or trans with respect to each other;
R¹ is —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;
R² is —H, halo, —CN, —NHR⁴, —NHC(O)R⁴, —NHC(O)OR⁴, —NHC(O)NHR⁴, —NHNHC(O)R⁴, —NHNHC(O)OR⁴, —NHNHC(O)NHR⁴, or —NH—N═C(R⁶)R⁷;
R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;
R⁶ is —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C3-C8 monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(C₃-C₈ monocyclic cycloalkenyl), -phenylene-(CH₂)ₙCOOH, or -phenylene-(CH₂)ₙCOO—(C₁-C₁₀ alkyl);
R⁷ is —H, —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkenyl) or —(CH₂)ₙ—(C₈-C₁₂ bicyclic cycloalkyl);
and each n is independently an integer ranging from 1 to 5, and a pharmaceutically acceptable vehicle.

2. A method of reducing age-related macular degeneration in a subject in need thereof, comprising the step of: applying an effective amount of an ophthalmic pharmaceutical composition comprising a compound according to Formula I to an affected eye of the subject, 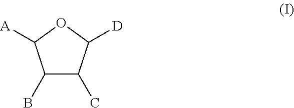
or a pharmaceutically acceptable salt thereof,
wherein
A is —CH₂ONO₂, —CH₂OH— or —CH₂OSO₃H;
B and C are —OH;
D is 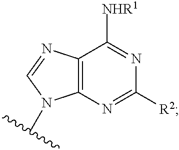
A and B are trans with respect to each other;
B and C are cis with respect to each other;
C and D are cis or trans with respect to each other;
R¹ is —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—C₈-C₁₂ bicyclic cycloalkenyl), or —(CH₂)ₙ-aryl;
R² is —H, halo, —CN, —NHR⁴, —NHC(O)R⁴, —NHC(O)OR⁴, —NHC(O)NHR⁴, —NHNHC(O)R⁴, —NHNHC(O)OR⁴, —NHNHC(O)NHR⁴, or —NH—N═C(R⁶)R⁷;
R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C3-C8 monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;
R⁶ is —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ-(C₃-C₈ monocyclic cycloalkyl), —(CH₂)ₙ—(C₃-C8 monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ—(C₃-C₈ monocyclic cycloalkenyl), -phenylene-(CH₂)ₙCOOH, or -phenylene-(CH₂)ₙCOO—(C₁-C₁₀ alkyl);
R⁷ is —H, —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkenyl) or —(CH₂)ₙ—(C₈-C₁₂ bicyclic cycloalkyl);
and each n is independently an integer ranging from 1 to 5, and a pharmaceutically acceptable vehicle.

3. A method of treating age-related macular degeneration in a subject in need thereof, comprising the step of: applying an effective amount of an ophthalmic pharmaceutical composition comprising a compound according to Formula I to an affected eye of the subject, 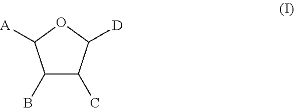
or a pharmaceutically acceptable salt thereof,
wherein
A is —CH₂ONO₂, —CH₂OH— or —CH₂OSO₃H;
B and C are —OH;
D is 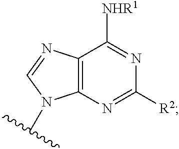
A and B are trans with respect to each other;
B and C are cis with respect to each other;
C and D are cis or trans with respect to each other;
R¹ is —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;
R² is —H, halo, —CN, —NHR⁴, —NHC(O)R⁴, —NHC(O)NHR⁴, —NHC(O)NHR⁴, —NHNHC(O)R⁴, —NHNHC(O)OR⁴, —NHNHC(O)NHR⁴, or —NH—N═C(R⁶)R⁷;
R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(_{CH2})ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;
R⁶ is —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ—(C₃-C₈ monocyclic cycloalkenyl), -phenylene-(CH₂)ₙCOOH, or -phenylene-(CH₂)ₙCOO—(C₁-C₁₀ alkyl);
R⁷ is —H, —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkenyl) or —(CH₂)ₙ—(C₈-C₁₂ bicyclic cycloalkyl);
and each n is independently an integer ranging from 1 to 5, and a pharmaceutically acceptable vehicle.

4. The method of claim 1, wherein the compound of Formula I has the formula: 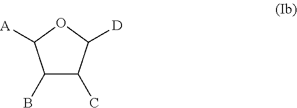
or a pharmaceutically acceptable salt thereof,
wherein
A is —CH₂ONO₂;
B and C are —OH;
D is 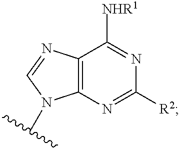
A and B are trans with respect to each other;
B and C are cis with respect to each other;
C and D are cis or trans with respect to each other;
R¹ is —C₃-C₈ monocyclic cycloalkyl, -3- to 7-membered monocyclic heterocycle or —C₈-C₁₂ bicyclic cycloalkyl; and
R² is —H or -halo.

5. The method of any claim 1, wherein the compound of Formula I is selected from: 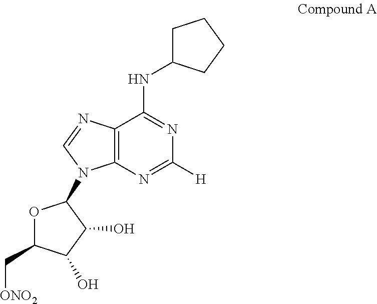
((2R,3S,4R,5R)-5-(6-(cyclopentylamino)-9H-purin-9-yl)-3,4-dihydroxytetrahydrofuran-2-yl)methyl nitrate, 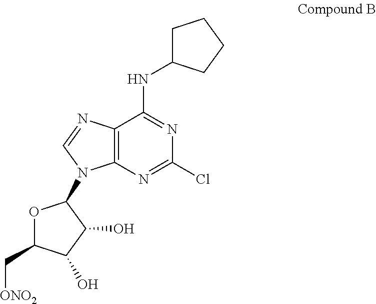
((2R,3S,4R,5R)-5-(2-chloro-6-(cyclopentylamino)-9H-purin-9-yl)-3,4-dihydroxytetrahydrofuran-2-yl)methyl nitrate, 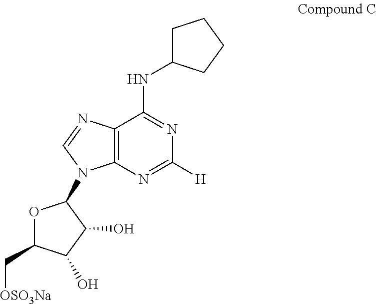
sodium ((2R,3S,4R,5R)-5-(6-(cyclopentylamino)-9H-purin-9-yl)-3,4-dihydroxytetrahydrofuran-2-yl)methyl sulfate, 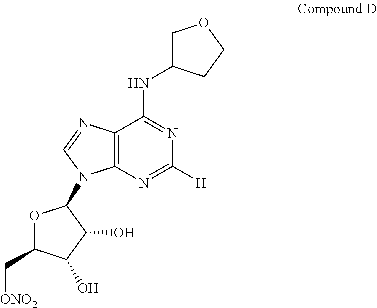
((2R,3S,4R,5R)-3,4-dihydroxy-5-(6-(tetrahydrofuran-3-ylamino)-9H-purin-9-yl)tetrahydrofuran-2-yl)methyl nitrate, 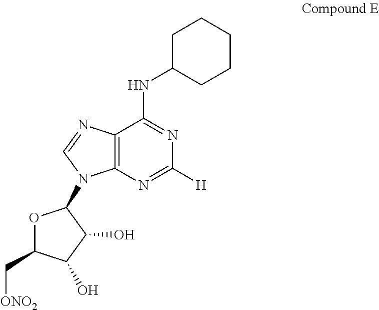
((2R,3S,4R,5R)-5-(6-(cyclohexylamino)-9H-purin-9-yl)-3,4-dihydroxytetrahydrofuran-2-yl)methyl nitrate, 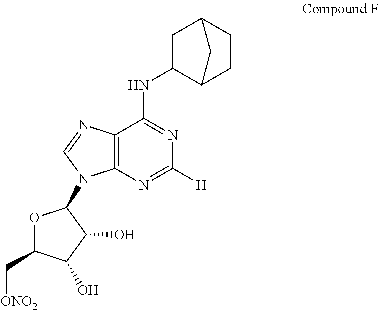
((2R,3S,4R,5R)-5-(6-(bicycle-[2.2.1]-heptan-2-ylamino)-9H-purin-9-yl)-3,4-dihydroxytetrahydrofuran-2-yl)methyl nitrate, 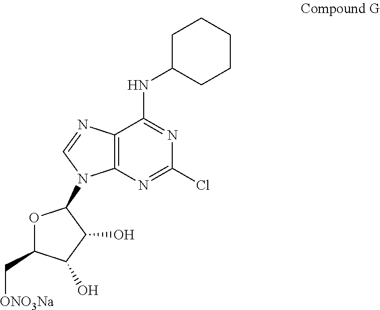
sodium ((2R,3S,4R,5R)-5-(2-chloro-6-(cyclohexylamino)-9H-purin-9-yl)-3,4-dihydroxytetrahydrofuran-2-yl)methyl sulfate, 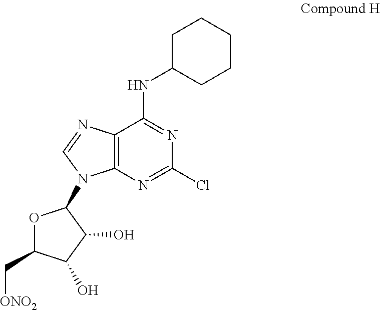
((2R,3S,4R,5R)-5-(2-chloro-6-(cyclohexylamino)-9H-purin-9-yl)-3,4-dihydroxytetrahydrofuran-2-yl)methyl nitrate, and 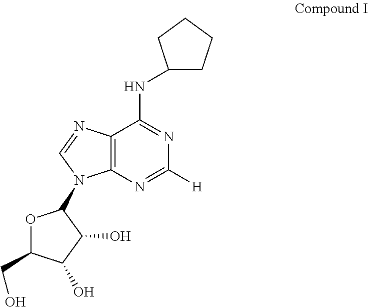
Cyclopentyladenosine (CPA), 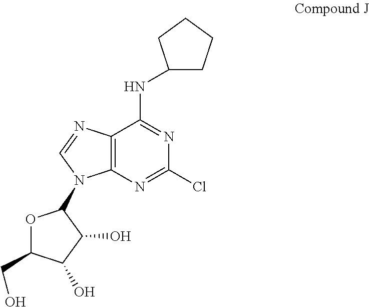
2-chlorocyclopentyladenosine (CCPA), 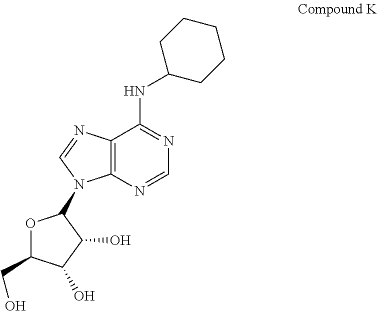
Cyclohexyladenosine (CHA),
or pharmaceutically acceptable salts thereof.

6. The method of claim 5, wherein the compound is selected from ((2R,3S,4R,5R)-5-(6-(cyclopentylamino)-9H-purin-9-yl)-3,4-dihydroxytetrahydrofuran-2-yl)methyl nitrate;
((2R,3S,4R,5R)-5-(2-chloro-6-(cyclopentylamino)-9H-purin-9-yl)-3,4-dihydroxytetrahydrofuran-2-yl)methyl nitrate;
sodium ((2R,3S,4R,5R)-5-(6-(cyclopentylamino)-9H-purin-9-yl)-3,4-dihydroxytetrahydrofuran-2-yl)methyl sulfate;
((2R,3S,4R,5R)-3,4-dihydroxy-5-(6-(tetrahydrofuran-3-ylamino)-9H-purin-9-yl)tetrahydrofuran-2-yl)methyl nitrate;
((2R,3S,4R,5R)-5-(6-(cyclohexylamino)-9H-purin-9-yl)-3,4-dihydroxytetrahydrofuran-2-yl)methyl nitrate;
((2R,3S,4R,5R)-5-(6-(bicycle-[2.2.1]-heptan-2-ylamino)-9H-purin-9-yl)-3,4-dihydroxytetrahydrofuran-2-yl)methyl nitrate;
sodium ((2R,3S,4R,5R)-5-(2-chloro-6-(cyclohexylamino)-9H-purin-9-yl)-3,4-dihydroxytetrahydrofuran-2-yl)methyl sulfate;
((2R,3S,4R,5R)-5-(2-chloro-6-(cyclohexylamino)-9H-purin-9-yl)-3,4-dihydroxytetrahydrofuran-2-yl)methyl nitrate; cyclohexyladenosine (CHA); 2-chlorocyclopentyladenosine (CCPA); and cyclopentyladenosine (CPA).

7. The method of claim 1, wherein the compound is Compound A ((2R,3S,4R,5R)-5-(6-(cyclopentylamino)-9H-purin-9-yl)-3,4-dihydroxytetrahydrofuran-2-yl)methyl nitrate.

8. The method of claim 1, comprising applying about 0.1% to about 5.0% (w/v) of the compound of Formula I from 1 to 4 times daily.

9. The method of claim 8, comprising applying about 1.0% to about 3.0% (w/v) of the compound of Formula I from 1 to 4 times daily.

10. The method of claim 8, comprising applying about 0.5% to about 1.5% (w/v) of the compound of Formula I from 1 to 4 times daily.

11. The method of claim 1, wherein the pharmaceutical composition is administered in drops.

12. The method of claim 11, wherein the pharmaceutical composition is administered in 1 to 2 drops.

13. A method of preventing, reducing or treating retinal pigment epithelium damage in a subject by administering a pharmaceutical composition comprising an effective amount of a selective adenosine A₁ agonist to an eye of the subject.

14. The method of claim 13, wherein the subject has or is at risk of developing age-related macular degeneration.

15. The method of claim 13, wherein the subject has or is at risk of developing dry age-related macular degeneration.

16. The method of claim 13, wherein the selective adenosine A₁ agonist is a compound of Formula I, 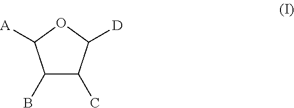
or a pharmaceutically acceptable salt thereof,
wherein
A is —CH₂ONO₂, —CH₂OH— or —CH₂OSO₃H;
B and C are —OH;
D is 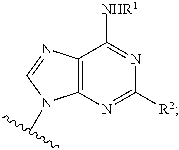
A and B are trans with respect to each other;
B and C are cis with respect to each other;
C and D are cis or trans with respect to each other;
R¹ is C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;
R² is —H, halo, —CN, —NHR⁴, —NHC(O)R⁴, —NHC(O)OR⁴, —NHC(O)NHR⁴, —NHNHC(O)R⁴, —NHNHC(O)OR⁴, —NHNHC(O)NHR⁴, or —NH—N═C(R⁶)R⁷;
R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;
R⁶ is —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ—(C₃-C₈ monocyclic cycloalkenyl), -phenylene-(CH₂)ₙCOOH, or -phenylene-(CH₂)ₙCOO—(C₁-C₁₀ alkyl);
R⁷ is —H, —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkenyl) or —(CH₂)ₙ—(C₈-C₁₂ bicyclic cycloalkyl) each n is independently an integer ranging from 1 to 5, and a pharmaceutically acceptable vehicle.

17. The method of claim 16, wherein the selective A₁ agonist is a compound of formula I, 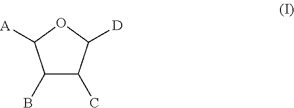
or a pharmaceutically acceptable salt thereof,
wherein
A is —CH₂ONO₂;
B and C are —OH;
D is 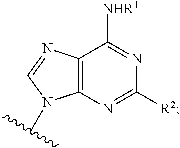
A and B are trans with respect to each other;
B and C are cis with respect to each other;
C and D are cis or trans with respect to each other;
R¹ is —C₃-C₈ monocyclic cycloalkyl, -3- to 7-membered monocyclic-heterocycle or —C₈-C₁₂ bicyclic cycloalkyl; and
R² is —H or -halo.

18. The method of claim 17, wherein the compound of Formula I is selected from: 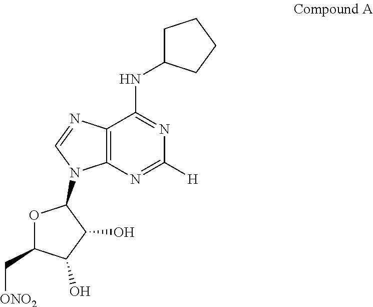
((2R,3S,4R,5R)-5-(6-(cyclopentylamino)-9H-purin-9-yl)-3,4-dihydroxytetrahydrofuran-2-yl)methyl nitrate, 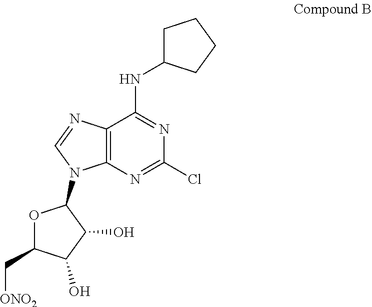
((2R,3S,4R,5R)-5-(2-chloro-6-(cyclopentylamino)-9H-purin-9-yl)-3,4-dihydroxytetrahydrofuran-2-yl)methyl nitrate, 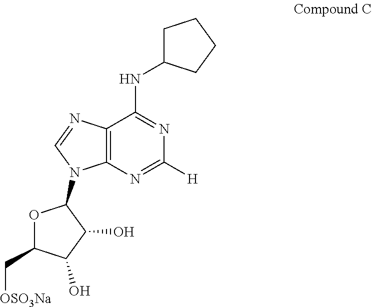
sodium ((2R,3S,4R,5R)-5-(6-(cyclopentylamino)-9H-purin-9-yl)-3,4-dihydroxytetrahydrofuran-2-yl)methyl sulfate, 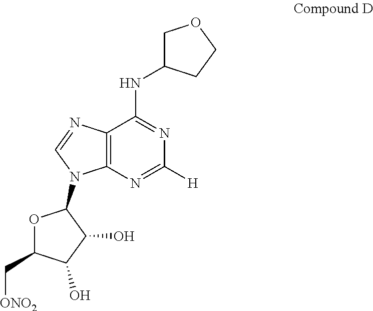
((2R,3S,4R,5R)-3,4-dihydroxy-5-(6-(tetrahydrofuran-3-ylamino)-9H-purin-9-yl)tetrahydrofuran-2-yl)methyl nitrate, 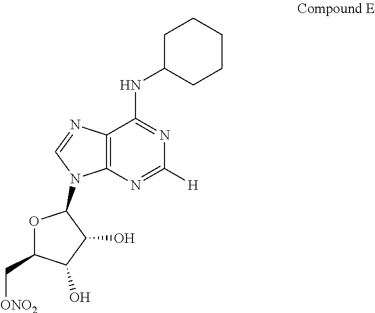
((2R,3S,4R,5R)-5-(6-(cyclohexylamino)-9H-purin-9-yl)-3,4-dihydroxytetrahydrofuran-2-yl)methyl nitrate, 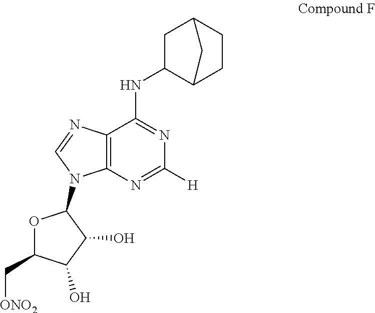
((2R,3S,4R,5R)-5-(6-(bicycle-[2.2.1]-heptan-2-ylamino)-9H-purin-9-yl)-3,4-dihydroxytetrahydrofuran-2-yl)methyl nitrate, 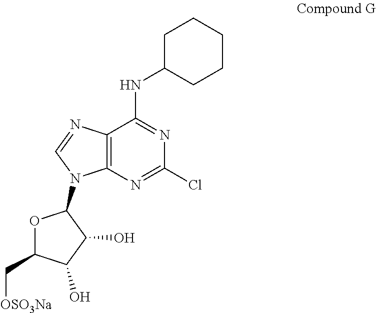
sodium ((2R,3S,4R,5R)-5-(2-chloro-6-(cyclohexylamino)-9H-purin-9-yl)-3,4-dihydroxytetrahydrofuran-2-yl)methyl sulfate, 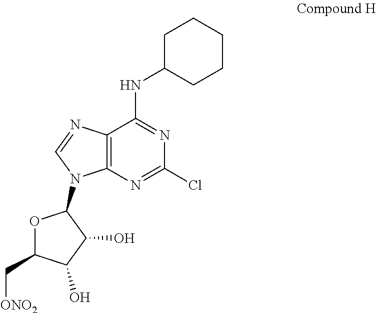
((2R,3S,4R,5R)-5-(2-chloro-6-(cyclohexylamino)-9H-purin-9-yl)-3,4-dihydroxytetrahydrofuran-2-yl)methyl nitrate, and 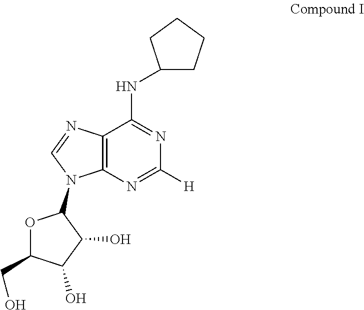
cyclopentyladenosine (CPA), 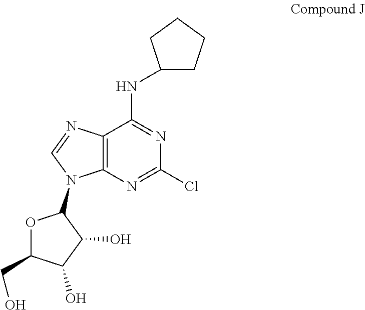
2-chlorocyclopentyladenosine (CCPA), 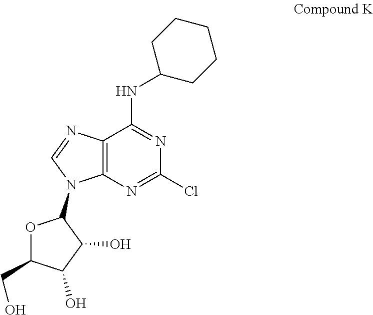
Cyclohexyladenosine (CHA),
or pharmaceutically acceptable salts thereof.

19. The method of claim 18, wherein the compound of Formula I selected is: 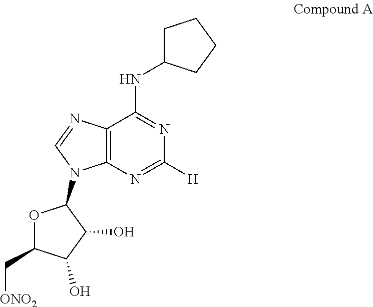
((2R,3S,4R,5R)-5-(6-(cyclopentylamino)-9H-purin-9-yl)-3,4-dihydroxytetrahydrofuran-2-yl)methyl nitrate; or a pharmaceutically salt thereof.

20. A method of preventing, reducing or treating photoreceptor cell damage in a subject by administering a pharmaceutical composition comprising an effective amount of a selective adenosine A₁ agonist to an eye of the subject.

21. The method of claim 20, wherein the subject has or is at risk of developing age-related macular degeneration.

22. The method of claim 21, wherein the subject has or is at risk of developing dry age-related macular degeneration.

23. The method of claim 20, wherein the selective adenosine A₁ agonist is a compound of Formula I, 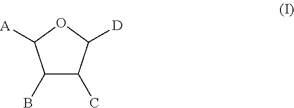
or a pharmaceutically acceptable salt thereof,
wherein
A is —CH₂ONO₂, —CH₂OH— or —CH₂OSO₃H;
B and C are -OH;
D is 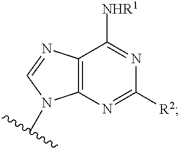
A and B are trans with respect to each other;
B and C are cis with respect to each other;
C and D are cis or trans with respect to each other;
R¹ is —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;
R² is —H, halo, —CN, —NHR⁴, —NHC(O)R⁴, —NHC(O)OR⁴, —NHC(O)NHR⁴, —NHNHC(O)R⁴, —NHNHC(O)OR⁴, —NHNHC(O)NHR⁴, or —NH—N═C(R⁶)R⁷;
R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ-(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;
R⁶ is —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ—(C₃-C₈ monocyclic cycloalkenyl), -phenylene-(CH₂)ₙCOOH, or -phenylene-(CH₂)ₙCOO—(C₁-C₁₀ alkyl);
R⁷ is —H, —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ-(C₃-C₈ monocyclic cycloalkyl), —(CH₂)ₙ—(C₃-C₈ monocyclic cycloalkenyl), —(CH₂)ₙ-(C₈-C₁₂ bicyclic cycloalkenyl) or —(CH₂)ₙ-(C₈-C₁₂ bicyclic cycloalkyl) each n is independently an integer ranging from 1 to 5, and a pharmaceutically acceptable vehicle.

24. The method of claim 23, wherein the selective A₁ agonist is a compound of formula 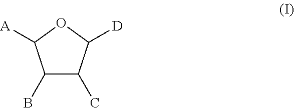
or a pharmaceutically acceptable salt thereof,
wherein
A is —CH₂ONO₂;
B and C are —OH;
D is 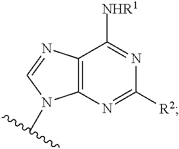
A and B are trans with respect to each other;
B and C are cis with respect to each other;
C and D are cis or trans with respect to each other;
R¹ is —C₃-C₈ monocyclic cycloalkyl, -3- to 7-membered monocyclic-heterocycle or —C₈-C₁₂ bicyclic cycloalkyl; and
R² is —H or -halo.

25. The method of claim 24, wherein the compound of Formula I is selected from: 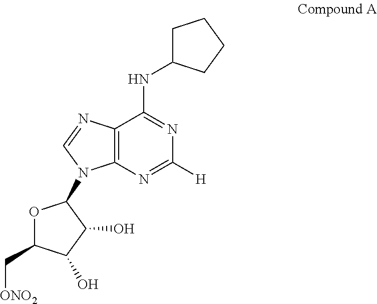
((2R,3S,4R,5R)-5-(6-(cyclopentylamino)-9H-purin-9-yl)-3,4-dihydroxytetrahydrofuran-2-yl)methyl nitrate, 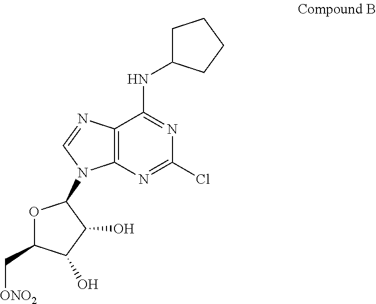
((2R,3S,4R,5R)-5-(2-chloro-6-(cyclopentylamino)-9H-purin-9-yl)-3,4-dihydroxytetrahydrofuran-2-yl)methyl nitrate, 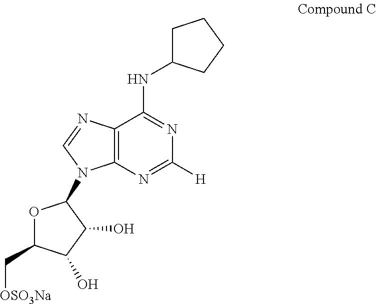
sodium ((2R,3S,4R,5R)-5-(6-(cyclopentylamino)-9H-purin-9-yl)-3,4-dihydroxytetrahydrofuran-2-yl)methyl sulfate, 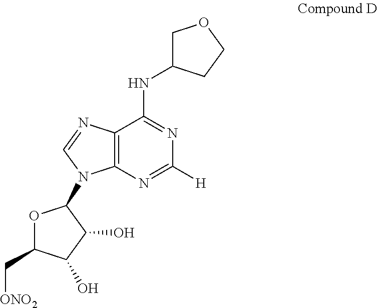
((2R,3S,4R,5R)-3,4-dihydroxy-5-(6-(tetrahydrofuran-3-ylamino)-9H-purin-9-yl)tetrahydrofuran-2-yl)methyl nitrate, 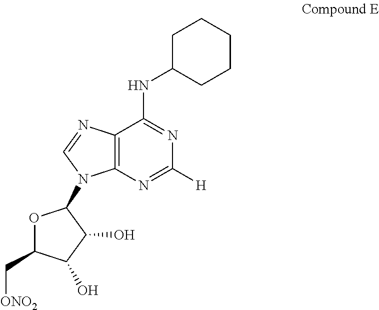
((2R,3S,4R,5R)-5-(6-(cyclohexylamino)-9H-purin-9-yl)-3,4-dihydroxytetrahydrofuran-2-yl)methyl nitrate, 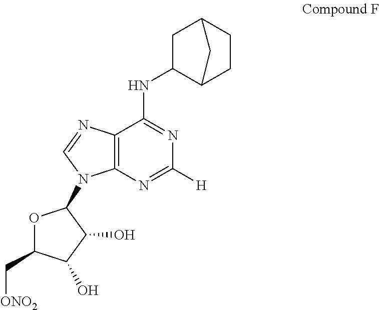
((2R,3S,4R,5R)-5-(6-(bicycle-[2.2.1]-heptan-2-ylamino)-9H-purin-9-yl)-3,4-dihydroxytetrahydrofuran-2-yl)methyl nitrate, 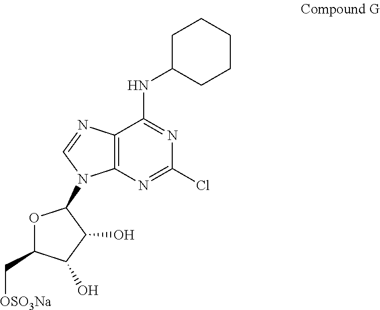
sodium ((2R,3S,4R,5R)-5-(2-chloro-6-(cyclohexylamino)-9H-purin-9-yl)-3,4-dihydroxytetrahydrofuran-2-yl)methyl sulfate, 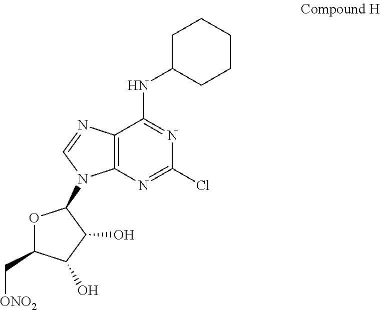
((2R,3S,4R,5R)-5-(2-chloro-6-(cyclohexylamino)-9H-purin-9-yl)-3,4-dihydroxytetrahydrofuran-2-yl)methyl nitrate, and 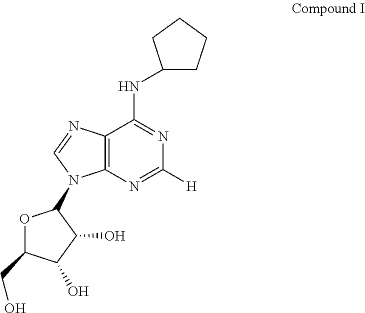
cyclopentyladenosine (CPA), 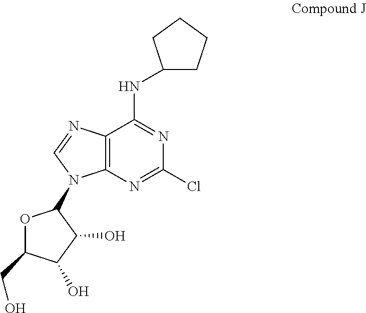
2-chlorocyclopentyladenosine (CCPA), 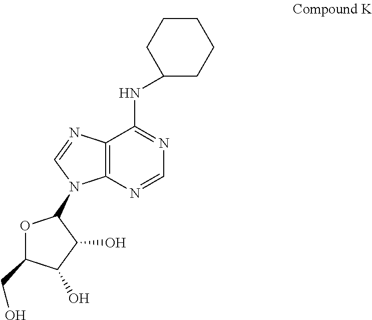
Cyclohexyladenosine (CHA),
or pharmaceutically acceptable salts thereof.

26. The method of claim 25, wherein the compound of Formula I selected is: 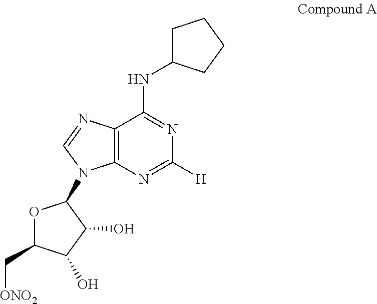
((2R,3S,4R,5R)-5-(6-(cyclopentylamino)-9H-purin-9-yl)-3,4-dihydroxytetrahydrofuran-2-yl)methyl nitrate; or a pharmaceutically acceptable salt thereof.
